(19) United States
(12) Patent Application Publication
(10) Pub. No.: US 2015/0057433 A1
(43) Pub. Date: Feb. 26, 2015

(54) PREPARATION OF FUNCTIONALIZED POLYPEPTIDES, PEPTIDES, AND PROTEINS BY ALKYLATION OF THIOETHER GROUPS

(71) Applicant: The Regents of the University of California, Oakland, CA (US)

(72) Inventors: Timothy J. Deming, Los Angeles, CA (US); Jessica R. Kramer, Los Angeles, CA (US)

(73) Assignee: The Regents of the University of California, Oakland, CA (US)

(21) Appl. No.: 14/388,777

(22) PCT Filed: Mar. 26, 2013
(86) PCT No.: PCT/US2013/033938

§ 371 Date: Sep. 26, 2014

Related U.S. Application Data
(60) Provisional application No. 61/615,809, filed on Mar. 26, 2012.

(51) Int. Cl. C07K 1/113

(52) U.S. Cl. CPC C07K 1/113
USPC 530/345; 530/402

(57) ABSTRACT

Reagents are disclosed for chemoselective tagging of methionine residues in peptides and polypeptides, subsequent bioorthogonal tag functionalization, and cleavage of the tags when desired to regenerate unmodified samples. This method compliments other peptide tagging strategies and adds capability for tag removal, which may be useful for release of therapeutic peptides from a carrier, or release of tagged protein digests from solid supports.

### CROSS-REFERENCE TO PRIOR APPLICATIONS

The present application is based on and claims the benefit and priority of U.S. Provisional Patent Application No. 61/615,809 filed on 26 Mar. 2012 which is incorporated herein by reference in its entirety to the extent such incorporation is permitted by law and regulation.

### U.S. GOVERNMENT SUPPORT

This invention was made with Government support under Grant No. MSN 1057970, awarded by the National Science Foundation. The Government has certain rights in this invention.

### BACKGROUND OF THE ART

1. Area of the Art

The present invention is related to chemical methods for modifying the amino acid residues of peptides and proteins and is more specifically related to a method of modification based on alkylation of thioether groups.

2. Description of the Background Art

There is considerable interest in the site specific conjugation of molecules, i.e. “tags”, to peptides and proteins (8). These tags may be used for attachment of probes for imaging, for selective purification or detection in complex mixtures, for enhancement of therapeutic properties, or as labels to assist in proteomic analysis (67). Such modifications typically rely on chemoselective reactions with natural amino acid functional groups, e.g. cysteine thiols (8), or biosynthetic incorporation of unnatural amino acids that present functionality for bioorthogonal reactivity, e.g. azide groups (66). While many approaches exist for selective tagging of peptides and proteins, few of these, aside from labile disulfides, are reversible modifications that allow triggered regeneration of the unmodified samples (8, 18, 19, 61, 66). Tag removal would be advantageous for some applications, such as release of therapeutic peptides from a carrier, or release of tagged protein digests from solid supports or affinity columns to allow downstream proteomic analysis (48, 58, 62, 72, 78 and 81). Here, we report the development of reagents for permanent or completely reversible, chemoselective alkylation of natural methionine (Met) residues or other thioether containing residues in peptides and polypeptides (Eq. 1). These reagents have been optimized to give stable sulfonium products, allow secondary modifications, and allow selective tag removal under mild conditions in certain cases.

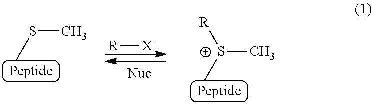

We have applied methods for alkylation of thioether functional groups to the synthesis new homo, random, and block polypeptides containing reactive functional groups that can be difficult to introduce into polypeptides, peptides and proteins in a controlled manner. Our strategy utilizes the naturally occurring thioether groups found in the amino acid methionine as a means to selectively modify polypeptides to introduce new, in some cases chemically reactive, functionality. Functional polymers are desirable for many applications, but are typically difficult to prepare due to the variable reactivity of the functional groups which can interfere with monomer preparation or polymerization. There has been much work recently on the post-polymerization functionalization of polymers that contain reactive functional groups. In particular, reactive polypeptides have been prepared utilizing a number of different functional groups introduced at both the monomer stage and on the polymers themselves, including ester, alkyl halide, alkyl azide, alkyne and alkene. All of these methods rely on introduction of unnatural functional groups by modification of amino acid monomers or polypeptides to create the reactive groups. Such approaches require additional synthetic steps, which can raise costs, lower yields, and can introduce additional linkers and functionalities that may not be desirable. Our approach takes advantage of the inherent and selective reactivity of methionine, a natural amino acid, which is considerably less expensive and easier to use compared to an unnatural or side chain functionalized amino acid. As such, we are able to create reactive polypeptides in fewer steps, using less expensive monomers compared to other approaches. Our approach can also be used for functionalization of methionine containing peptides and proteins, which broadens its utility. The polypeptides described here are readily prepared and production can be scaled to large quantities, making these materials economically viable. These new functional polypeptides have potential use in applications including therapeutics, diagnostics, antimicrobials, delivery vehicles, coatings, composites, and regenerative medicine.

There have been many examples where polypeptides have been chemically modified to improve their properties for various applications. Typically, this strategy has involved the hydrophobic modification of poly(lysine) or poly(glutamate/aspartate) side-chains by covalent attachment of lipophilic groups. These modifications are akin to polymer grafting reactions and thus result in random placement of these hydrophobic substituents (typically long alkyl chains) along the polypeptide backbone. These modifications were often performed in order to increase the polypeptide's ability to bind hydrophobic drugs, aggregate in aqueous solution, and/or penetrate the lipid bilayers of cell walls. The random placement of the hydrophobic groups along the chains meant that they cannot act as distinct domains in supramolecular assembly, as in a block copolymer, thus limiting their ability to form ordered structures.

Other types of chemical polypeptide modification include the addition of non-ionic, polar groups to increase solubility and blood circulation lifetime, addition of mesogenic groups, addition of linker groups to allow efficient functionalization of pre-formed polypeptides via “click” reactions, and the addition of sugars. Increasing bioavailability and biofunctionality are major goals for development of useful synthetic polypeptide materials. One approach to synthesis of functional polypeptides is the synthesis of monomers that contain the desired functionality, which can then be polymerized to give corresponding functional polypeptides. This strategy avoids difficulties associated with derivatization of polymers, and allows for 100% residue functionalization, but can be limited economically by lengthy multistep monomer syntheses and difficulties in monomer purification and polymerization. As an alternative strategy, many groups have pursued the use of fast, efficient “click” type reactions to attach functionality to pre-formed polypeptides.

Examples of this strategy include the work of Hammond (17) who reported using copper catalyzed coupling of functionalized azides to alkyne functional homopolypeptides synthesized from synthetic γ-propargyl-L-glutamate N-carboxyanhydride (NCA). This method has been used to attach amine, polyethyleneglycol, and monosaccharide groups to this polypeptide. In a related strategy, Zhang (74) reported the preparation of γ-3-chloropropyl-L-glutamate NCA and its corresponding polymer, which was further modified by conversion of chloro to azido groups that were then coupled to alkyne functionalized D-mannose using copper catalysis. Heise (30) also synthesized poly(D/L-propargylglycine) from the NCA of the commercially available amino acid, and then coupled azide functionalized galactose to this polypeptide using copper catalysis.

Utilizing a different type of click reaction, Schlaad (71) prepared poly(D/L-allyglycine) from the commercially available amino acid to directly incorporate alkene groups into polypeptides for thiol-ene coupling. Radical addition of a variety of thiols, catalyzed by AIBN at elevated temperature using two equivalents of thiol per alkene gave polymers with degrees of alkene functionalization that varied greatly with reaction conditions. Using this methodology, ester and monosaccharide functionality was added to the poly(D/L-allyglycine) segments. Cheng (50) has also recently reported the synthesis of a reactive polypeptide synthesized from γ-(para-vinylbenzyl)-L-glutamate NCA. This polymer undergoes a variety of reactions either through the pendant alkene group, or through its conversion to a benzaldehyde functionality. While promising, these methods can suffer from incomplete functionalization, as well as incorporation of unnatural groups, such as triazole or benzyl that may limit biological uses. Many of these methods also require synthesis of an unnatural functional monomer that requires additional cost, additional synthetic steps, and difficulties in monomer purification and polymerization.

Our lab recently reported the use of Met alkylation as a facile means to chemoselectively introduce useful functionality and chemically reactive groups onto polypeptides (45). This work was based on the pioneering studies of Met alkylation in proteins, which were mainly focused on use of non-functional alkylating reagents to probe inhibition of enzyme active sites (4, 25, 29, 38, 47, 57, 68 and 79). While many of these alkylations were reported to be irreversible (4, 25, 29, 47, 68 and 79), some studies (38 and 57), as well as subsequent experiments with peptides (5, 16, 28, 56, 59, and 73), found that Met alkylation can be reversed under certain conditions. Since each study typically employed a different substrate (amino acid, peptide or protein), different alkylating reagents, and different nucleophilic cleavage reagents (4, 5, 16, 25, 28, 29, 38, 47, 56, 57, 59, 68, 73 and 79), comparison of reactivity and properties of the various alkylated Met sulfonium groups that have been reported is challenging. This is especially true since Met alkylations with some reagents (e.g. benzylic halides)(68, and 73) have been reported to be reversible in some cases and irreversible in others (5, 38, and 64). As a further complication, reaction of Met sulfoniums with nucleophiles can give other products, aside from regenerating Met, depending on where nucleophilic attack occurs (28). In light of these uncertainties, we sought to undertake a systematic study to evaluate Met sulfonium stability as functions of both alkylating reagent and added nucleophile using a model copolypeptide substrate. Since there have been no reports on chemical modification of reversible Met alkylating tags, we also sought to develop tags with this unprecedented feature. Our goal was to identify optimized reagents and conditions for stable sulfonium formation, introduction of bioorthogonal reactive groups for tag modification, and facile removal of the functional tags when desired.

Here we disclose the preparation of a wide variety of new functionalized polypeptide compositions by the straightforward reaction of functional group containing alkylating reagents with homopolymers and copolymers containing naturally occurring methionine. In terms of prior art, the alkylation of thiol (i.e. sulfhydryl) groups in polymers and polypeptides to introduce new functionality has been described, however, this is a different process entirely since thiol and thioether groups have very different chemical stabilities and reactivities. More related to our disclosure, the alkylation of various synthetic polymers containing thioether groups to give polysulfonium salts has also been previously disclosed. However, these reactions were performed only to introduce cationic sulfonium ions and improve water solubility. The prior examples did not attempt or demonstrate the addition of a broad range of reactive or new functional groups since alkylations were limited to only hydrophobic alkyl (methyl, ethyl, benzyl) and hydrophilic carboxymethyl groups. The prior art also did not mention or demonstrate any chemoselectivity in the alkylation procedure. Only two forms of alkylation (namely methylation or carboxymethylation) of polymethionine or other synthetic methionine containing copolypeptides have been previously described, which were also pursued to obtain water soluble polypeptides. Methionine amino acids, short methionine containing peptides (<10 residues), or proteins have also been alkylated using a wider variety of alkylating agents, which were all based primarily on either bromoacetyl or iodoacetyl derivatives.

Considering the prior art in this area, the alkylation of methionine or S-alkyl cysteine residues in synthetic random, block or homopolypeptides (>5 residues long) using groups other than methyl or carboxymethyl has not been previously described. Also noteworthy, there are no reports of alkylation of methionine residues in any peptide, protein or polypeptide using alkylating agents with structures other than iodomethane, bromomethane, benzyl bromide, cyanogen bromide, diethyl bromomalonate, or the bromoacetyl or iodoacetyl derivatives XCH2C(O)R, where X═Br or I, and R═OH, OEt, NH₂, C₆H₆, NH(substituted phenyl), NH(L-fucose), or NH(D-galactose). The alkylation of multiple methionine residues in a single polypeptide chain is also unprecedented. While the concept of alkylation of methionine residues in peptides, proteins, and polypeptides, using only alkyl halides, has been demonstrated, our work here is inventive since we have improved upon and expanded the scope of existing methods for methionine alkylation, in turn also creating new compositions of matter in the form of alkylated methionine containing polypeptides containing previously unanticipated, and importantly chemically reactive, functional groups. This approach and these compositions are novel as there is no existing prior art that shows or suggests the possibility or advantages of adding such functionality or reactive groups to methionine residues in polypeptides, peptides or proteins. We also have improved the methods for alkylation of methionine containing polypeptides to allow their modification by a wider range of alkylating reagents than had been previously described or considered.

### DESCRIPTION OF THE FIGURES

FIG. 1 shows (FIG. 1a) Molecular weight (Mₙ, ♦) as a function of monomer to initiator ratio ([M]/[I]) for poly(Met) prepared by polymerization of Met NCA using (PMe₃)₄Co in THF at 20° C.; and FIG. 1b) GPC chromatogram (normalized LS intensity versus elution time in arbitrary units (a.u.)) of glycopolypeptide 15, M_{w}/Mₙ=1.14.

FIG. 2 is a table showing results of alkylation of poly(Met); reagents and conditions: R—X in DMF, H₂O, or 0.2 M aqueous formic acid, 20° C.; yield is total isolated yield of completely functionalized polypeptide; [a] product was dialyzed against 0.1 M aqueous NaCl to give X═Cl. [b] X═Br. [c] X═I.

FIG. 3 is a table showing results of alkylation of poly(Met) using AgBF₄; reagents and conditions: R—X, AgBF₄, MeCN, 50° C.; yield is total isolated yield of completely functionalized polypeptide; [a] product was dialyzed against 0.1 M aqueous NaCl to give X═Cl. [b] product was dialyzed against aqueous HCl at pH=2 with 0.1 M NaCl to give the polyketone, 14a, and X═Cl. [c] X═BF₄.

FIG. 4 is a table showing results of alkylation of poly(Met) using alkyl triflates; reagents and conditions: R-OTf, DCM/MeCN, 20° C.; yield is total isolated yield of completely functionalized polypeptide; [a] X=OTf. [b] product was dialyzed against 0.1 M aqueous NaCl to give X═Cl.

FIG. 5 is a diagram showing Scheme 1 for chemoselective alkylation of methionine residues in the presence of excess amine groups, followed by clicking of PEG-N₃ to the alkylated methionine residues; initial copolymer is poly[(N_{ε}-TFA-L-lysine)_{0.8}-stat-(Met)_{0.2}]₂₀₆, where 0.8 and 0.2 are mole fractions of lysine and methionine residues, respectively; reagents and conditions: (a) K₂CO₃, MeOH, H₂O (99%); (b) propargyl bromide, 0.2M formic acid (94%); (c) α-methoxy-ω-azidoethyl-poly(ethylene glycol) (Mₙ=1,000 Da), CuSO₄, ascorbic acid, PMDETA, H₂O (95%).

FIG. 6 shows some specific examples of some additional functional and reactive alkylating reagents according to the present invention.

FIG. 7 shows structures of KM substrate, alkylating reagents (R—X), and nucleophiles (Nuc); GSH=glutathione; reagent 2e was reacted with a Met homopolymer instead of KM.

FIG. 8 are graphs showing the results of dealkylation of polymers 3b, 3c, and 3g over time using different Nuc (0.1 M in PBS, 37° C.); FIG. 8A uses PyS and FIG. 8 B uses GSH.

FIG. 9 is a schematic showing tag, modify, and release studies on KM copolypeptide.

FIG. 10 shows MALDI-MS spectra of (FIG. 10A) PHCRKM (M⁺) 21, (FIG. 10 B) PHCKRM alkylated with 2g to give 25 (MR⁺), and (FIG. 100) 25 after treatment with PyS to regenerate PHCRKM; M(O)R⁺ represents some 25 that had oxidized during MS ionization.

FIG. 11 shows graphs of regeneration of KM from polysulfoniums over time using different nucleophiles (37° C., PBS buffer); FIG. 11A uses 0.1 M 2-mercaptoethanol; FIG. 11B uses 0.1 M thiourea; and FIG. 11C uses 0.1 M 2-mercaptopyridine.

FIG. 12 shows the chemical reactions for regeneration of KM from 3g using 0.1 M 2-mercaptopyridine (37° C., PBS buffer) and structure of isolated reaction byproduct.

FIG. 13 shows fluorescence spectra of polypeptide 3g: initially (3g), after copper catalyzed attachment of azidofluorescein 4c (5c), and after dealkylation using mercaptopyridine to give parent polypeptide KM; (FIG. 13A=absorption spectra; and FIG. 13 B=emission spectra).

FIG. 14 shows ¹H NMR spectra (all are 2 mg/mL in D₂O) of (FIG. 14A) PHCKRM; (FIG. 14B) PHCKRM regenerated from 21 after treatment with PyS; and (FIG. 14C) alkylated PHCKRM, 21, which is a mixture of diasteromers due to sulfonium chirality.

FIG. 15 shows expanded MALDI-MS spectra of (FIG. 15A) PHCRKM, (FIG. 15B) PHCKRM alkylated with 2g to give 21, and (FIG. 15C) 21 after treatment with PyS to regenerate PHCRKM; negligible multiply alkylated products were observed.

FIG. 16 shows a MALDI-MS spectrum of PHCKRM alkylated with 2g at pH 8.3; multiple alkylated products are observed.

FIG. 17 shows ESI-MS detection of HPLC samples of (FIG. 17A) PHCKRM; and (FIG. 17B) PHCKRM regenerated after treatment of 21 with PyS with positive ionization.

FIG. 18 shows GPC chromatograms (normalized LS intensity versus elution time in arbitrary units (au)) of copolypeptides after initial copolymerization of Met and CBz-Lys NCAs and endcapping with PEG-NCO to give 22 (−), alkylation with 2g to give polysulfonium 23 ( . . . ) and after dealkylation of the sulfonium groups using mercaptopyridine to regenerate the parent 22 (- - - ).

### DETAILED DESCRIPTION OF THE INVENTION

The following description is provided to enable any person skilled in the art to make and use the invention and sets forth the best modes contemplated by the inventor of carrying out his invention. Various modifications, however, will remain readily apparent to those skilled in the art, since the general principles of the present invention have been defined herein specifically to provide methods for chemically modifying thioethers in peptides and polypeptides

This invention includes the introduction of various functional groups onto polypeptides by alkylation of thioether (a.k.a. sulfide) groups, creating new compositions of matter. The thioether groups may either be present in the polypeptides, or may be added to polypeptides containing thioether precursors, such as thiol, alkene or alkyl halide functional groups. We have used existing methods, as well as developed new methods, for alkylation of thioether groups in polypeptides. The methods used are general and can be applied to a wide range of different peptidic materials, including polypeptides, peptides and proteins. Examples of this invention are the modification of polypeptides via the thioether groups naturally present in methionine or in S-alkyl cysteine residues. A variety of new, and particularly important, chemically reactive functionalities have been added to polypeptides via this process, including alkenes, alkynes, boronic acids, sulfonates, phosphonates, alkoxysilanes, carbohydrates, secondary, tertiary, quaternary and alkylated amines, pyridines, alkyl halides, and ketones, creating many new functional polypeptides, each of which are new compositions of matter. This alkylation process is a simple one-step modification, and is also chemically selective, allowing one to introduce chemically reactive functionalitiy to specific locations on polypeptides, peptides, and proteins. It is thus an economical way to prepare polypeptides with complex functionality that have potential use in applications including therapeutics, diagnostics, antimicrobials, delivery vehicles, coatings, composites, and regenerative medicine.

We recently developed a flash column chromatography method that enabled the high yield, straightforward purification of L-methionine NCA (Met NCA). This monomer is infrequently used in polypeptide synthesis since it is typically obtained as a non-crystallizable syrup that is difficult to purify, and its controlled polymerization has only recently been demonstrated. Met NCA purified by chromatography, obtained as a white solid, was successfully polymerized in our lab using (PMe₃)₄Co in THF at monomer to initiator (M:I) ratios up to 200 to 1 with complete conversion to poly(L-methionine) of controllable lengths (equation 2). In our lab, Met NCA was also copolymerized with Z-Lys NCA to form statistical or block copolypeptides that were soluble in DMF and could be analyzed by GPC/LS. Polymerization of equimolar mixtures of Met NCA and Z-Lys NCA at different M:I ratios gave statistical copolypeptides whose lengths (Mₙ) increased linearly with M:I stoichiometry, and which possessed narrow chain length distributions (M_{w}/Mₙ). Stepwise polymerization of Met NCA and Z-Lys NCA under similar conditions afforded the block copolymers, which are new compositions of matter. These data showed the first instance where well-defined poly(L-methionine) and methionine containing copolypeptides were prepared, and have facilitated the possibility of using polymethionine segments in new biopolymer materials.

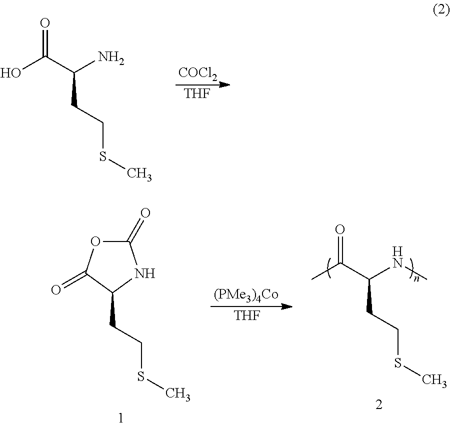

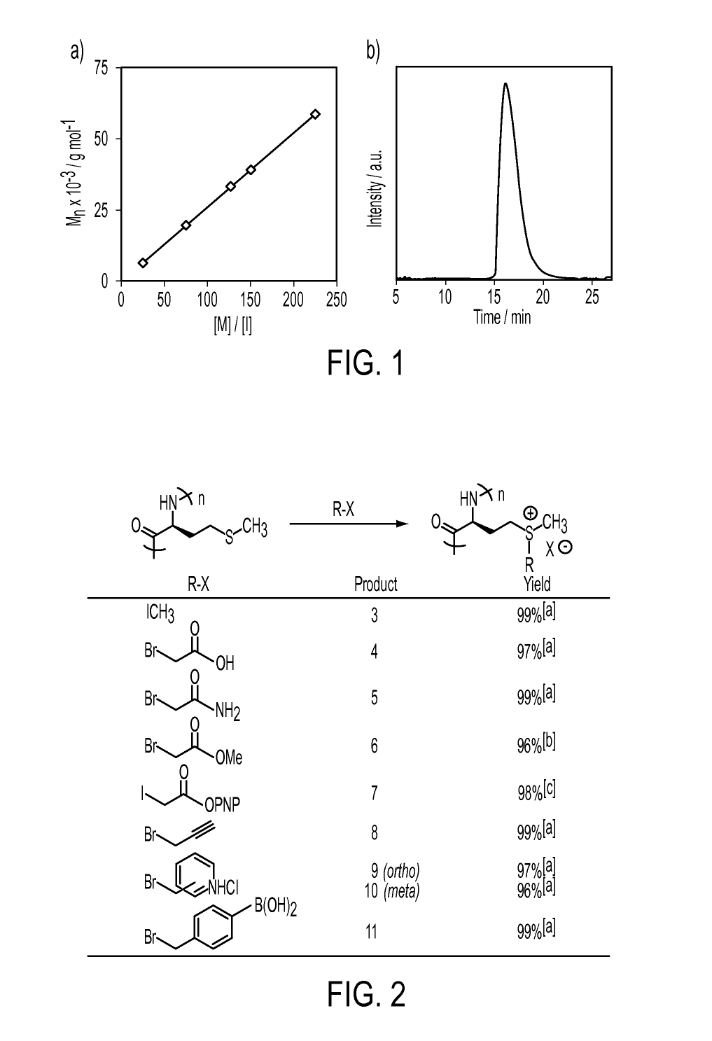

Homopolymerizations of Met NCA (eq 2) go to completion within a few hours at room temperature, yet molecular weight analysis of these chains is difficult due to aggregation of poly(Met) in solution. To determine chain lengths, Met NCA was polymerized at different monomer to (PMe₃)₄Co initiator ratios, and after complete monomer consumption, active chains were end-capped with isocyanate terminated PEG (Mₙ=2000 Da). Compositional analysis of purified, end-capped polymers by ¹H NMR gave average poly(Met) chain lengths that increased linearly with stoichiometry (FIG. 1). Although chain length distributions of these poly(Met) samples could not be obtained, GPC analysis of an alkylated poly(Met) (vide infra) was found to possess a narrow polydispersity index (M_{w}/Mₙ) of 1.14, indicating the parent poly(Met)s are also well-defined (FIG. 1). Poly(Met) was prepared in high yield with precisely controlled chain lengths up to over 400 residues long, and could also be prepared as statistical and block copolymers with other amino acids (see experimental section). Overall, these data show that Met NCA, similar to other NCAs, is able to undergo living polymerization when initiated with (PMe₃)₄Co.

Methionine has been generally considered to be a hydrophobic, non-reactive amino acid in peptides and proteins, and only recently have efforts been made to better understand its role in biology. Similarly, unmodified poly-L-methionine is a hydrophobic, α-helical polypeptide that has limited solubility in some organic solvents, including dichloromethane, formic acid, and trifluoroacetic acid, but is not soluble in water. Although α-helical poly(Met) has low solubility in most solvents, it is soluble enough to allow facile alkylation in a variety of different media. In our initial studies, poly(Met) was reacted with a variety of alkylating reagents in either DMF, deionized water, or 0.2 M aqueous formic acid. We observed that only activated alkyl bromides and iodides were able to react efficiently with poly(Met) under mild conditions (FIG. 2). Aside from the previously described products 3 and 4, other haloacetyl derivatives reacted readily and quantitatively to generate polysulfoniums bearing amide (27, 50, 65, and 71), ester (74) and active ester (30) functionalities, where the new compounds 6 and 7 are chemically reactive may be useful when further derivatized by reaction with nucleophiles, such as primary amines, which will provide a number of new compositions containing whatever functionality is also present on the amine reagent (e.g. polymer, drug, sugar, peptide, oligonucleotide, probe molecules, etc.). Propargylic and benzylic/pseudo-benzylic halides also reacted efficiently with poly(Met), and allow the introduction of a variety of useful functional groups into polypeptides. This method for introduction of alkyne functionality (25) is straightforward and more economical than other routes to install this click reactive group onto polypeptides. These alkyne containing polypeptides can be easily further modified by reaction with organic azides (R—N₃) with copper catalysis to introduce different functionalities in high yield. Since this reaction tolerates many different R—N₃ molecules, a tremendous range of functional groups may be introduced onto polypeptide using these alkyne modified methionines. Our results here demonstrate the first introduction of alkyne functionality onto methionine residues, using a process that competes well with other means to introduce alkyne group onto peptides, proteins or polypeptides. The facile reaction of benzylic and/pseudo-benzylic halides with methionine is a reaction that has hardly been explored. The only known example is the reaction of proteins with non-functional, hydrophobic benzyl bromide. Here, we show this reaction can be expanded to allow formation of pyridine containing sulfoniums (2, 53, 75, and 76), which allows incorporation of this basic functionality that is otherwise difficult to introduce in polypeptides. Phenyl boronic acid containing polypeptides (29, and 79) have also been of interest for their sugar-binding abilities, and now we have demonstrated these can be readily prepared with high degrees of incorporation in a single step. The functionalization of methionine using other ring-substituted benzyl halides is also envisioned, which will include active ester groups to allow reaction with functionalized primary amines, hydroxy groups to include phenolic and catechol functionalities, and active carbonates to allow formation of functional carbamates.

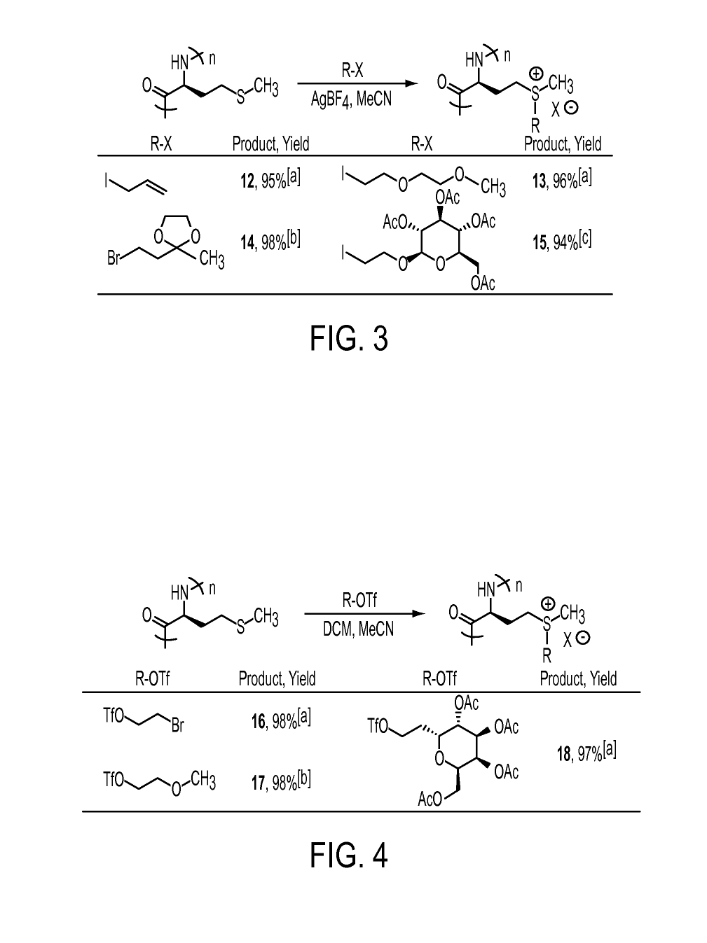

In contrast to the data reported above, unactivated alkyl halides, especially those without adjacent multiple bonds, reacted either sluggishly or not at all with poly(Met) under similar conditions. To further expand the scope of methionine alkylation, we developed new methods to increase reactivity of these alkyl halides with methionine. Silver tetrafluoroborate is known to promote thioether alkylation in small molecules, and we found that addition of this reagent to alkylations in acetonitrile promoted the complete alkylation of poly(Met) by unactivated alkyl halides (e.g. haloethyl compounds) (FIG. 3). This strategy allowed the facile incorporation of alkene functionality (64) onto polypeptides, which is useful for a variety of further modifications including thiol/ene click reactions, where a wide variety of functional thiol reagents (RSH) can be added to this group. Ethylene glycol derivatives (51, and 67) were also added to impart the water solubilizing and passivating properties of PEG. Reactive ketone groups, useful for conjugation in aqueous environments to a wide variety of amine, hydrazine and oxyamine reagents (i.e. RNH₂, RNHNH₂, RONH₂) to give functional derivatives, were introduced in a single step using the 1,3-dioxolane derivative (6, and 60) that deprotects to give the water soluble polyketone during acidic workup. Glycopolypeptides, of interest as mimics of natural glycoproteins, often require many synthetic steps, especially for preparation of longer chains with high sugar content. Using readily prepared iodoethyl glycosides, well defined, fully glycosylated polypeptides (43) were readily prepared in high yield by methionine alkylation.

Since the use of silver salts may not be desirable in some applications, we also developed the functionalization of poly(Met) using other alkylating reagents. Alkyl triflates are known to be powerful alkylating agents, and we discovered that these can react efficiently with poly(Met) as well. Functional alkyl triflates were prepared in a straightforward manner from a variety of hydroxyethyl compounds. These reagents reacted efficiently with poly(Met) in organic solvents under mild conditions to give the fully alkylated polymers (FIG. 4). Due to the significant difference in reactivity between alkyl triflates and bromides, this method allowed incorporation of alkyl bromide functionality onto poly(Met) (36). This electrophilic functionality can be readily modified by further reaction with different nucleophiles, such as amines, alcohols or thiols (i.e. RNH₂, ROH, RSH), to give a variety of functionalized polypeptides. As an example, we reacted polysulfonium 16 with aminomethane sulfonic acid, which gave quantitative incorporation of sulfonate functionality (40, and 55) that may be useful in mimicking sulfonated biopolymers (eq 2). Other functional groups that required silver salts for introduction onto poly(Met) could also be introduced by use of the corresponding alkyl triflates. Thus, PEG-like (7) and glycoside (14, and 15) functionalities were added to poly(Met) via the corresponding alkyl triflates. Removal of the acetyl protecting groups from the glycosylated polypeptide 18 gave a water soluble glycopolypeptide with no signs of any degradation (see experimental). In general, all of the above poly(Met) alkylations were found to cause no polypeptide chain cleavage, and gave polysulfoniums that were stable in a variety of media, at different pH (2 to 10), at elevated temperature (80° C.), and after storage for more than 3 months (see experimental).

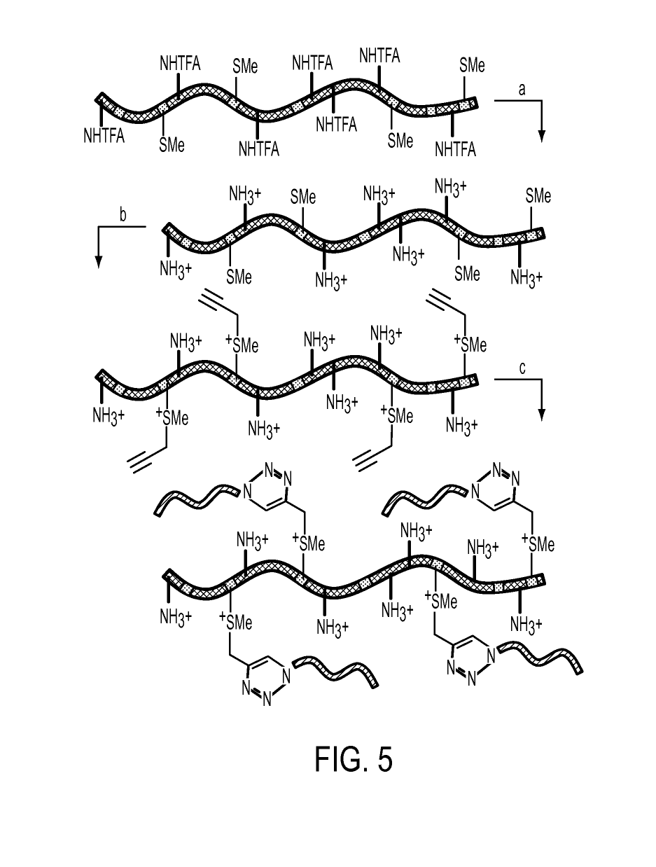

The results above confirm that alkylation of poly(Met) is a highly efficient process for preparation of functionalized polypeptides. The reaction is broad in scope, high yielding, occurs readily, can use equimolar reagents, has a single reaction trajectory, gives stable products, and purification of products is facile. It also allows a secondary functionalization since some of the functional groups that were introduced have the ability to react with a wide range of other functional molecules to give new derivatives in high yield (eq 3). However, to be considered a “click” reaction, it must also be chemoselective. In peptides and proteins, there are many nucleophilic functional groups that react with alkylating reagents. Of these, all except methionine exist in protonated forms at low pH, which greatly decreases their reactivity. While alkylations of proteinaceous functional groups, such as thiols, are common practice, methionine is the only functional group in proteins known to react with alkylating reagents at low pH. To confirm this selectivity, we prepared a statistical copolymer of methionine and lysine, the most common nucleophile found in proteins, and studied its alkylation (see Scheme 1 in FIG. 5). The synthesis of poly(L-lysine) requires the use of protecting groups, and the methionine residues of our copolymer could be alkylated with propargyl bromide either before or after lysine deprotection to give the same product (see experimental). These results show that the methionine sulfonium groups are stable to, and compatible with, deprotection reactions, and also that methionine residues can be alkylated chemoselectively in the presence of a fourfold excess of free amine groups. Confirming that only methionines were alkylated, a control reaction of propargyl bromide with pure polyp lysine) under identical conditions gave no alkylation products (see experimental). All the alkyne groups in the copolymer prepared above were then quantitatively conjugated in a secondary reaction with azide terminated PEG chains (see Scheme 1 in FIG. 5), showing that alkyne reactivity is not compromised and that the sulfonium linkages are stable to further reactions.

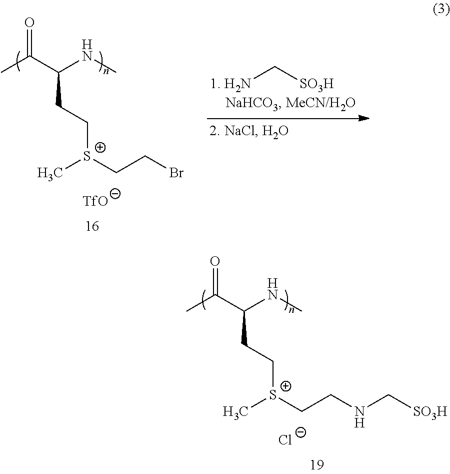

Possible Modifications to Process.

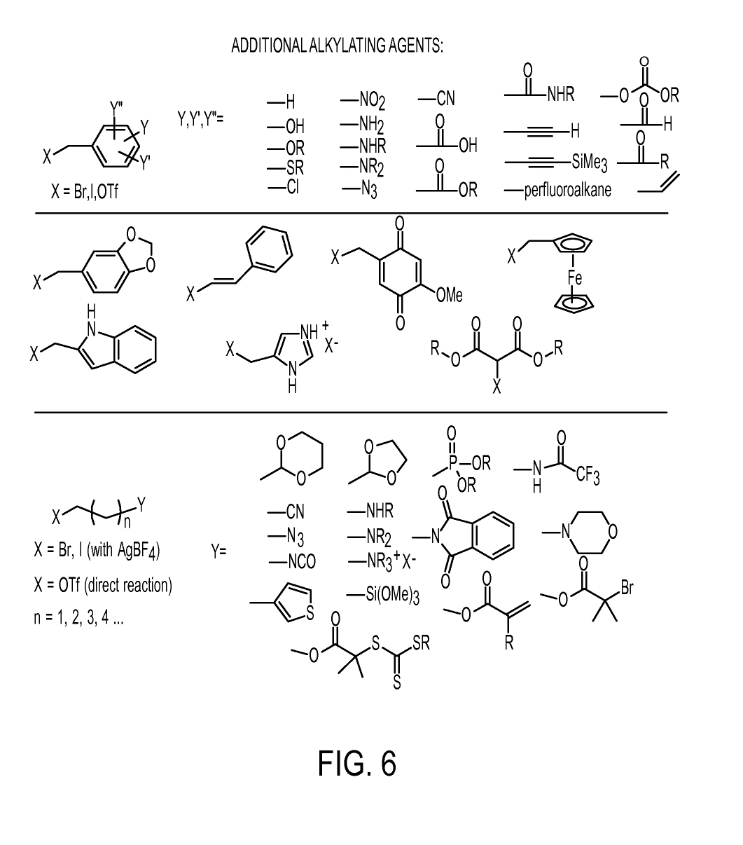

Our process of creating new reactive and functional polypeptide compositions as described above is very flexible with regard to many parameters including: nature of the alkylating agent, polypeptide composition (percentage of methionine in the polymers, peptide or proteins), use of other thioether containing polypeptides (e.g. S-alkyl cysteines), polypeptide architecture (block or random), use of D- or L-amino acids in the polymers, and conjugation of the polypeptide segments to other synthetic polymers. Also important, one can envision adding functionality to thioether groups found in other synthetic polymers, as these functional groups are readily created from widely used thiol-ene conjugation reactions. Such alkylations of these thioether groups have not been reported. In addition to the specific examples mentioned above, other alkylating agents or alkylation processes could be used to create similar functionalized polypeptides. In particular, other XCH₂C(O)R reagents, where X═Br or I; other benzylic/pseudo-benzylic bromides, iodides, or triflates; alkyl triflates of the general structure RCH₂CH₂OTf (typically prepared from commonly available (RCH₂CH₂OH); Alkyl bromides or iodides of the general structure RCH₂CH₂X, where X═Br or I, (R=functional or reactive residue) would all be considered to fall under the scope of this invention. Some specific examples of other alkylation reagents that fit these parameters are given in FIG. 6

Advantages Over Existing Methods.

Although there is some prior art on the alkylation of polymethionine, proteins, and short methionine containing peptides, our use of alkylation of thioether groups in polypeptides as a means for the introduction of a diverse array of reactive and useful functional groups onto polypeptides was not anticipated. We have also developed new alkylation methods to expand the scope of the types of functional groups that can be added to thioether containing polypeptides via alkylation. All prior work on polysulfonium synthesis was focused only on improvement of water solubility and introduction of the cationic sulfonium groups, i.e. to obtain polyelectrolytes. Hence, all prior work is very limited in scope, and limited to a narrow range of alkylating agents and peptidic (and other polymeric) materials. Here, we describe the preparation of new alkylated polypeptides, specifically many new alkylated polymethionine compositions, containing valuable functional groups that can be chemically reactive or useful in applications for their inherent properties. We have also developed the methods to apply this strategy to a wide range of other copolypeptides and polymers whose alkylation has not been reported. Our functional polypeptides are advantageous since they utilize a natural amino acid for the functionalization reaction, which is both significantly more cost effective and may impart better biocompatibility to the polymers compared to other approaches. The alkylation reaction itself is chemoselective and can be performed in the presence of other functional amino acid residues, the polymethionine precursor as well as alkylated products are stable to chemistries used to deprotect other functional groups (not true in many other methods to prepare polypeptides with reactive side-chain functional groups), and gives high yields of functionalization with a broad range of reagents. Our process allows inexpensive polymethionine to be used as a universal precursor polymer to prepare a wide range of functionalized polypeptide derivatives. The addition of reactive groups by alkylation also adds the ability to perform a secondary modification to the polypeptides to utilize an even broader range of selective reactions, e.g. “click” type reactions, that can take advantage of biocompatible reaction conditions. This capability may prove extremely useful for site-specific modification of methionine residues in peptides and proteins, that may be used in diagnostic devices or as therapeutics. The ability to introduce new functionality into a wide range of thioether containing groups in peptides, proteins, and polypeptides via this simple, and selective, process, now allows the economical preparation of a diverse array of new peptidic molecules with many potential applications including encapsulation, conjugation to, or delivery of therapeutics, foods, cosmetics, and agricultural products, as surface coatings, as antimicrobials, as tissue engineering scaffolds and biomaterials; as immunomodulators (e.g. vaccines, adjuvants); as well as imaging and diagnostic applications.

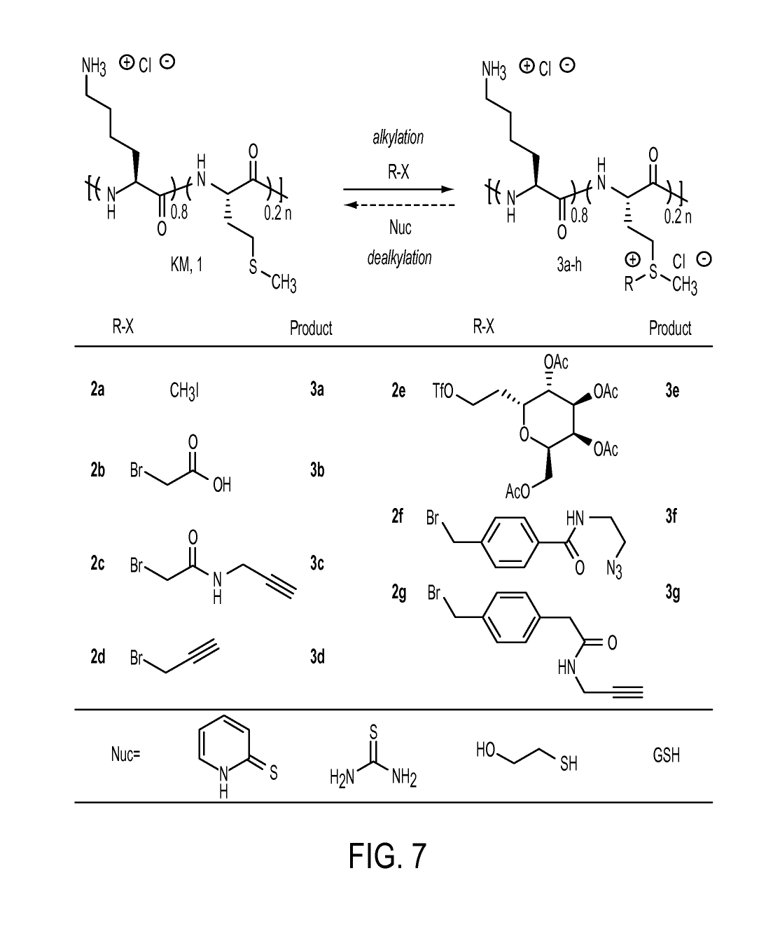

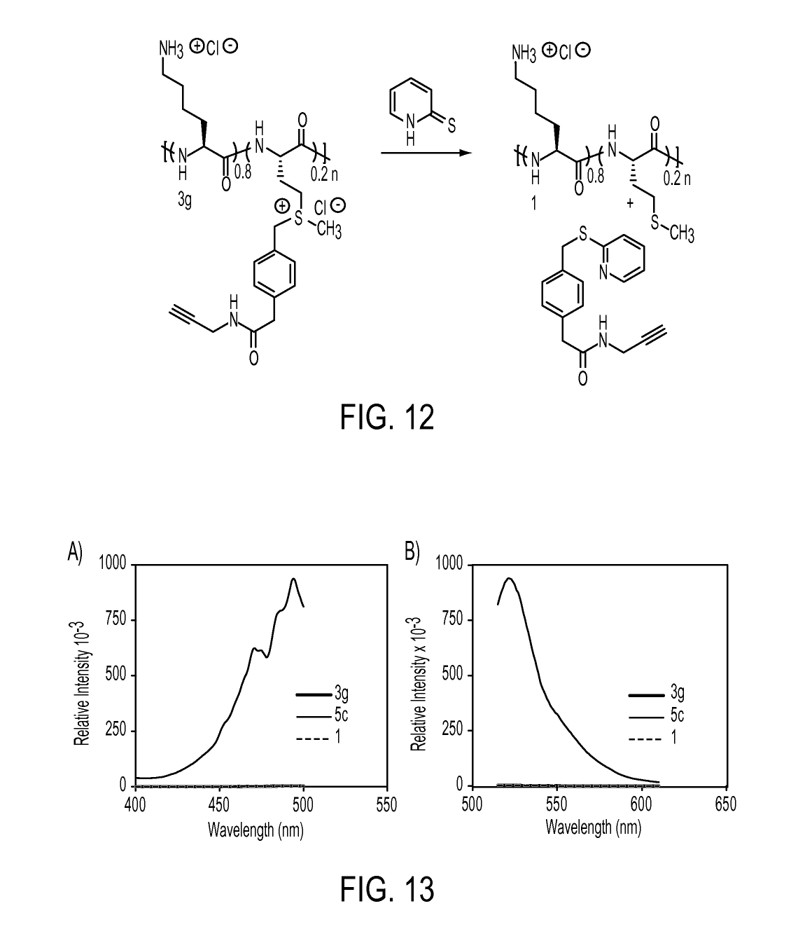

To study the stability of different Met sulfonium groups, we prepared a statistical copolymer of Met and lysine (KM, 1) and treated this model substrate separately with a variety of alkylating reagents (FIG. 7). Under the acidic conditions employed, all the Met residues in KM were chemoselectively alkylated in near quantitative yields, similar to previous results (45). Note that since peptides and polypeptides are routinely handled and manipulated in strongly acidic media, the acidic alkylation conditions are compatible with these molecules (18, 19 and 64). The resulting sulfonium containing copolymers were each then reacted with four common sulfur nucleophiles as shown in FIG. 7. We chose alkylating agents to cover a range of properties. The methyl (2a) and carboxymethyl (2b) groups were chosen as controls with non-reactive side-chains, and their sulfoniums 3a and 3b were found to be stable to all four nucleophiles, as well as strong base (pH 10) and heat (80° C.) in water (see FIG. 12). Although some reports state that these sulfoniums can dealkylated (5 and 38), our results are consistent with many studies that show these groups to be inert under similar conditions (4, 25, 29, 47, 68, and 79). The reagents 2c, 2d, and 2g were chosen to introduce desirable alkyne functionality that is useful for subsequent modification of the tagged copolypeptides under bioorthogonal conditions (66). An azide containing analog (2f) was also used to showcase the ability to incorporate different reactive groups, and finally a galactose containing reagent (2e) was used to introduce a model biofunctional side-chain.

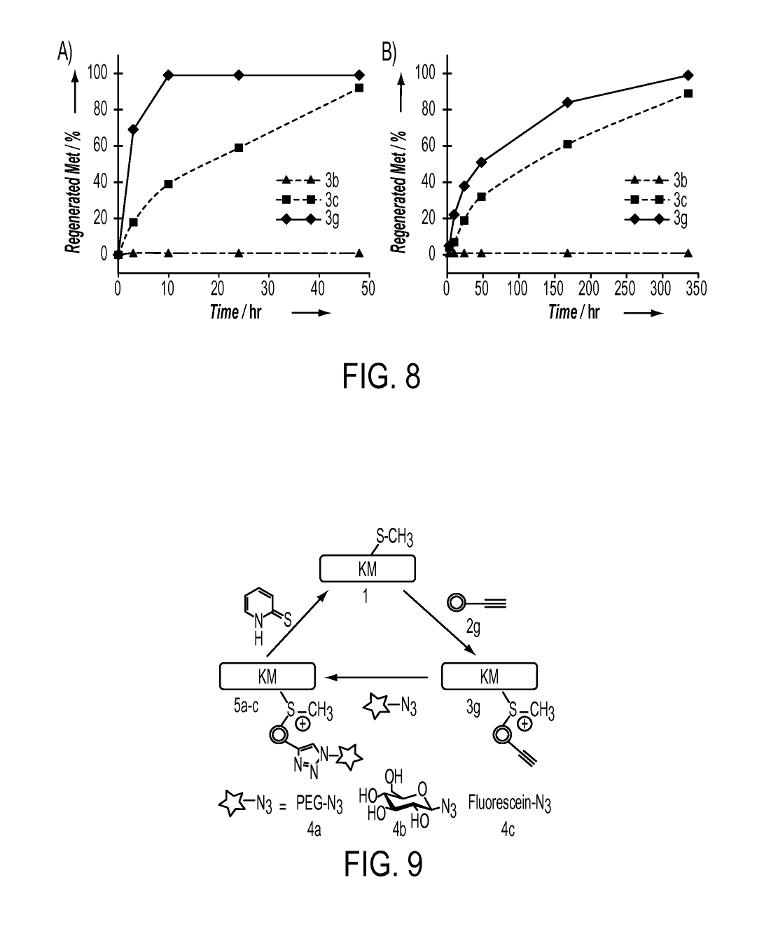

In summary, the alkylation of methionine residues in polypeptides has all the features of a “click” reaction, and consequently is an attractive general means for preparation of a wide range of functionalized polypeptides. Aside from the examples given here, this reaction should also be applicable to a variety of other alkylating reagents and thioether compounds, such as S-alkyl cysteines. The mild reaction conditions employed, especially for activated alkyl halides (FIG. 8), mean that this process is suitable for functional alkylation of methionine residues in peptides and proteins. In comparison to other polypeptide click reactions, methionine is substantially less expensive than side chain modified or unnatural amino acids, and poly(Met) requires minimal steps to prepare, making these “methionine click” reactions attractive for large-scale use. Facile incorporation of other click-reactive functional groups (e.g. alkyne or alkene) also allows for further chemoselective modification of methionine residues. Such “double click” strategies, as shown in Scheme 1 (FIG. 5), allow methionine alkylation to utilize the broad diversity of reagents already developed and available for other click conjugations.

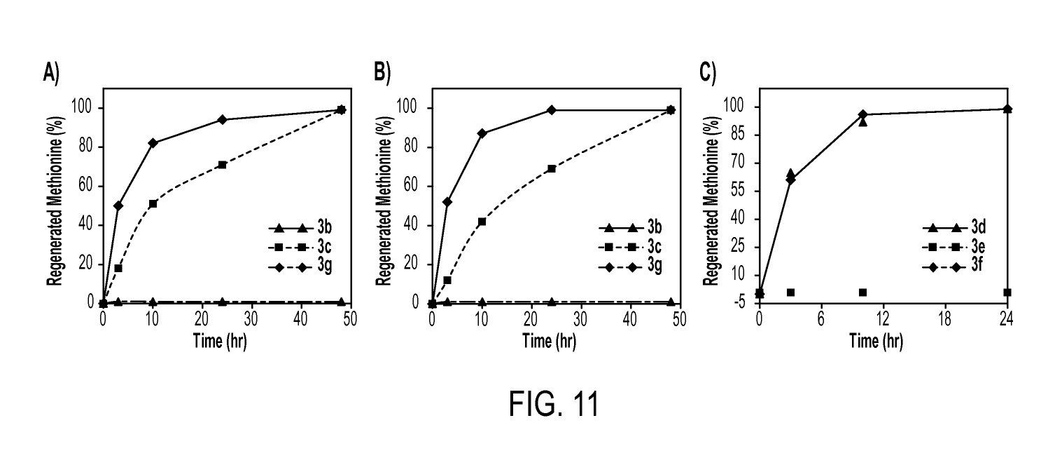

Upon treatment with sulfur nucleophiles, the copolypeptides 3c, 3d, 3f, and 3g all showed some dealkylation back to parent KM as the sole product, while glycopolymer 15 was found to be completely stable under these conditions (FIGS. 8 and 11). The stability of 15, like 3a, is most likely due to the lack of an electron withdrawing substituent on the alkylating carbon, resulting in the sulfonium being less electrophilic. The alkylating carbons of samples 3c, 3d, 3f, and 3g all have an activating substituent (carbonyl, alkyne or phenyl), which greatly increases the reactivity of these sulfoniums with nucleophiles. Glutathione (GSH) was found to be the least reactive nucleophile, but was eventually able to give high yields of dealkylated KM over time (FIG. 8 and below), which is relevant for applications in vivo. 2-Mercaptoethanol, thiourea, (31) and 2-mercaptopyridine (PyS) were all effective for quantitative dealkylation of sulfonium groups to regenerate KM (FIGS. 8 and 11), and PyS was chosen as the reagent of choice since it provides rapid sulfonium dealkylation, gives only a single byproduct, and also shows low reactivity with disulfides (see FIG. 12). While excess nucleophile was used in the studies described above, stoichiometric PyS was also found to effect quantitative sulfonium dealkylation with longer reaction times (see results, below).

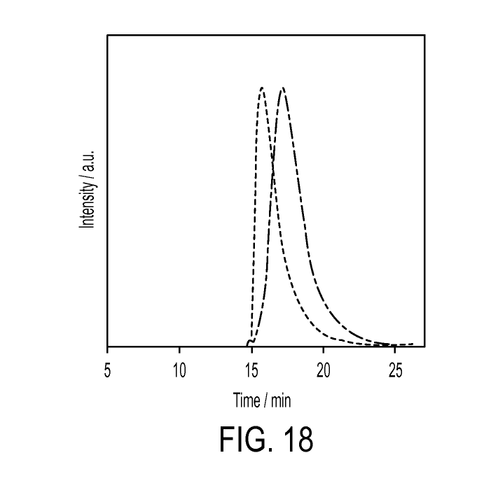

To identify optimal alkylating reagents, we focused on the alkyne containing polymers 3c, 3d, and 3g, which differ in linkage structure. While all of these copolypeptides were quantitatively dealkylated by PyS back to KM as the sole product, it was found that 3c was less desirable since it reacted much slower compared to 3d and 3g (FIGS. 8 and 11). The propargyl sulfonium 3d also had drawbacks since it was found to be unstable in basic aqueous media and upon prolonged storage as a solid (see below), and since its copper catalyzed cycloadditions with azides were sluggish. Consequently, the benzylic sulfonium derivatives 3f and 3g, were chosen since they provide an excellent combination of facile formation, stability against hydrolysis (pH 10), and rapid, facile dealkylation back to KM when treated with PyS. It is also worth noting that 3g was found to be completely stable in PBS buffer at 20° C. for 2 weeks, and that no peptide chain cleavage was detected after alkylation and dealkylation reactions (see FIG. 18).

To showcase the potential of this optimized system, we performed some proof of concept tag, modify, and release studies using the copolypeptide 3g (FIG. 9). A sample of 3g was prepared from KM as described above, and its alkyne tags were then modified via copper catalyzed cycloadditions using a variety of functionalized azides (66). Polyethylene glycol (PEG) chains and glucose units, which may be useful for improving biological lifetimes of peptide based therapeutics (63), were quantitatively attached to all alkylated Met residues in 3g (FIG. 9). As a model probe, 5-azidoacetamido-fluorescein was also attached to 3g (ca. 1 per polypeptide chain), to give the fluorescent derivative 5c (see FIG. 13). Treatment of each of these derivatives 5a, 5b, or 5c with PyS resulted in their quantitative conversion back to parent KM, confirming the facile release of the modified tags. We envision that a wide variety of azide, alkyne, or cycloalkyne containing molecules or substrates could be used to modify methionine containing peptide samples that have been tagged with one of the alkylating reagents 2f or 2g, making this chemistry attractive for applications when eventual release of the modified tag is desired.

For broad utility in tagging of peptides, Met alkylation needs to be a chemoselective process that is compatible and doesn't interfere with other peptide functional groups. In peptides and proteins, there are many nucleophilic functional groups that can react with alkylating reagents (35). Of these, all except Met exist in protonated forms at low pH, which greatly decreases their reactivity ([49). While alkylations of proteinaceous functional groups, such as thiols, are common practice at high pH (29), Met is the only functional group in proteins able to react with alkylating reagents at low pH. 15 (13, 23, 56, 57, and 77).

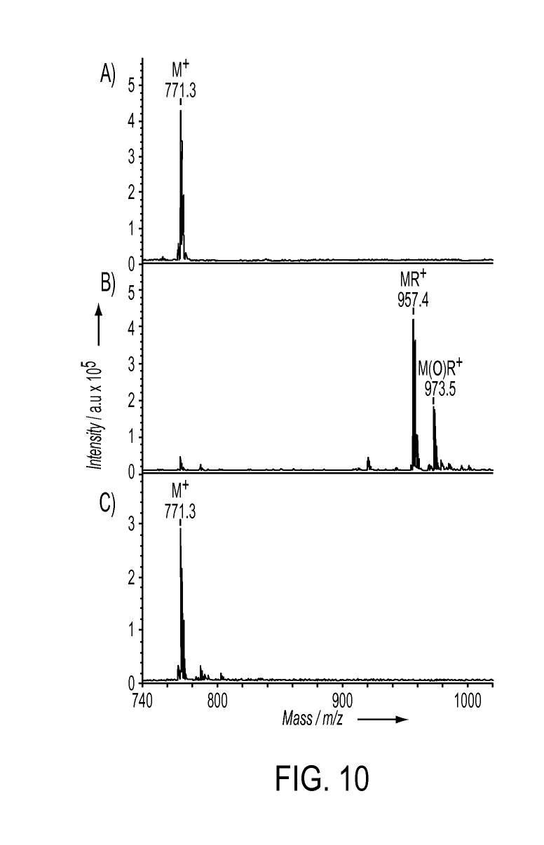

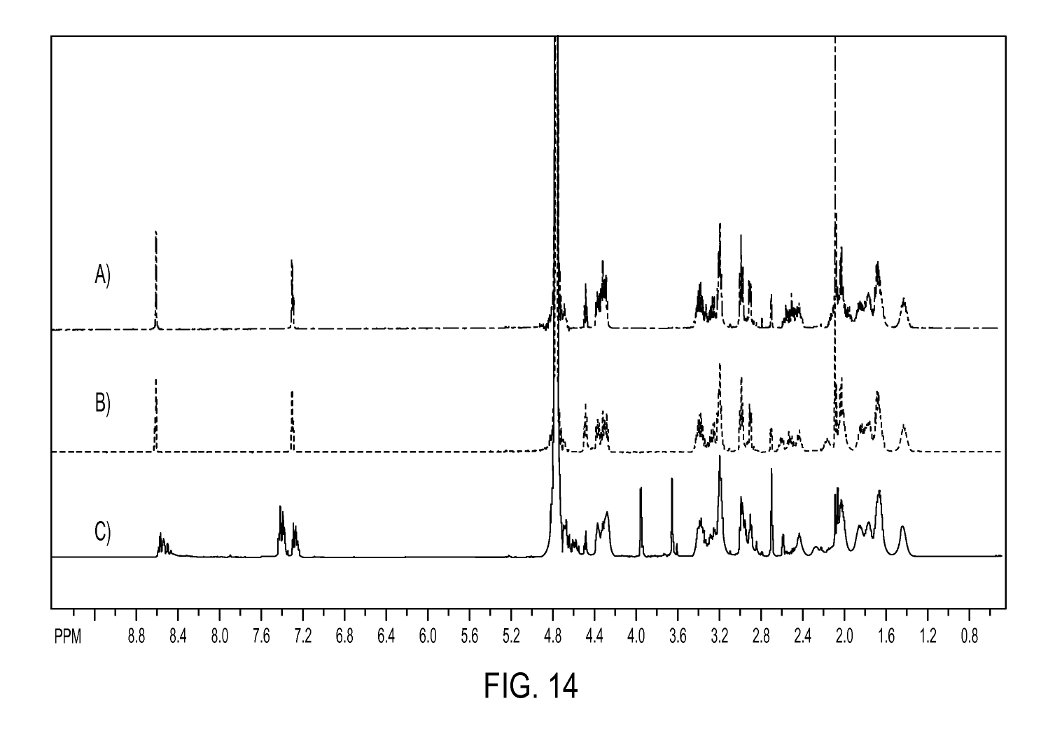

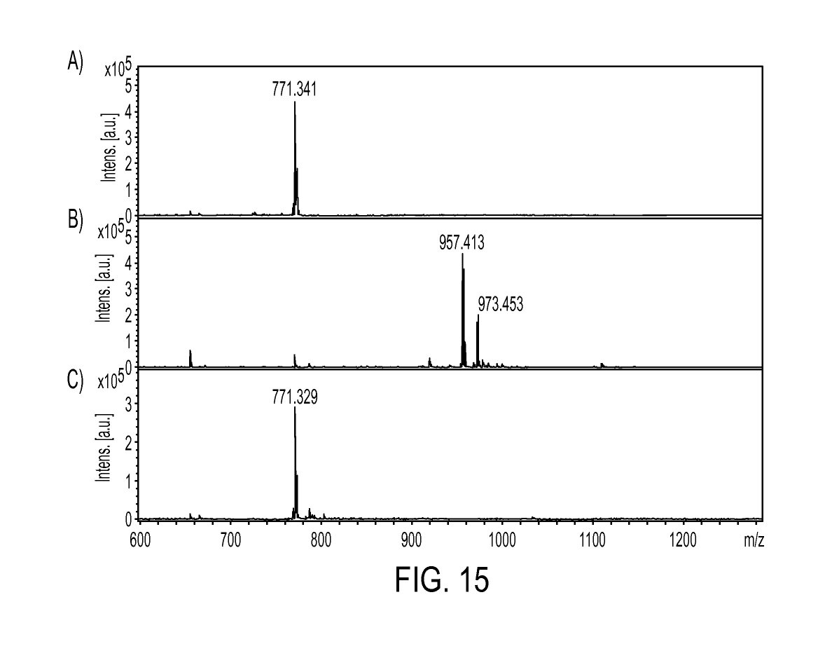

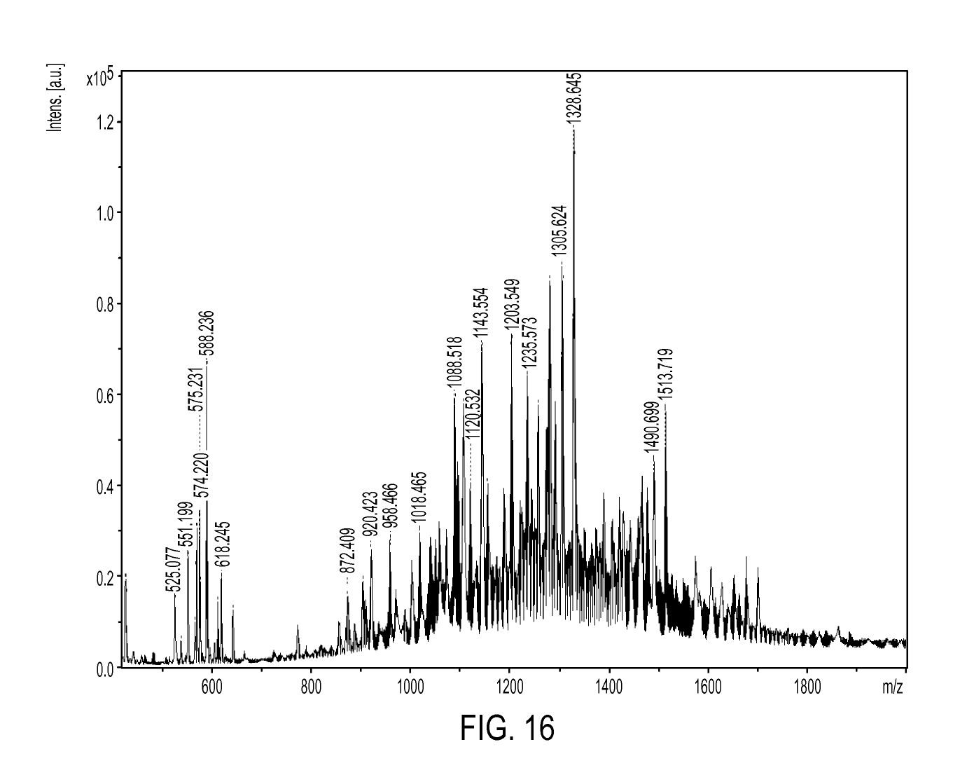

To demonstrate this selectivity, we attempted to alkylate only the Met residues in the antioxidant peptide PHCKRM, which also contains highly nucleophilic histidine, cysteine and lysine residues (Eq. 4). Treatment of PHCKRM with alkylating agent 2g in 0.2M aqueous formic acid (pH 2.4) gave only a single product 21 in 92% isolated yield, where only the Met residue was alkylated. The composition of 21 was confirmed using MALDI MS (FIG. 10), as well as 1H NMR analysis (see FIGS. 14 and 15), where the addition of a single 186 Da 2g tag in MS, and shift of the Met methyl resonance in NMR, were observed. The presence of the additional peak in B) at m/z 973 is indiciative of oxidation (addition of a single oxygen, Δm/z 16) of alkylated 21 during MALDI laser ionization. The oxidation is not at the Met residue since this is alkylated (m/z 973, not m/z 786 expected for Met sulfoxide of PHCKRM), and is likely due to oxidation at cysteine or histidine. The MALDI and ESI-MS spectra of dealkylated PHCKRM, as well as the ¹H NMR of 21 also show no evidence of oxidation, indicating that the oxidation seen in B) occurs only during MALDI MS analysis. Some oxidation (Δm/z 16) was observed in the MALDI MS spectrum of Met alkylated 21, which is likely at cysteine or histidine and is known to occur during MALDI ionization. 36-38 Control experiments where Na—Z-histidine, Na—Z-lysine, and Na—Z-cysteine were each reacted with benzyl bromide at pH 2.4 also showed no alkylation under these conditions. For contrast, reaction of PHCKRM with 2g (2 eq) in carbonate buffer (pH 8.3) showed the formation of a complex mixture of multiply alkylated peptides (see FIG. 16). These results demonstrate that peptides containing a variety of nucleophilic natural amino acid side-chains can be chemoselectively, and near quantitatively, modified at Met residues at low pH, as compared to the less selective, yet widely used alkylation of cysteines at higher pH.

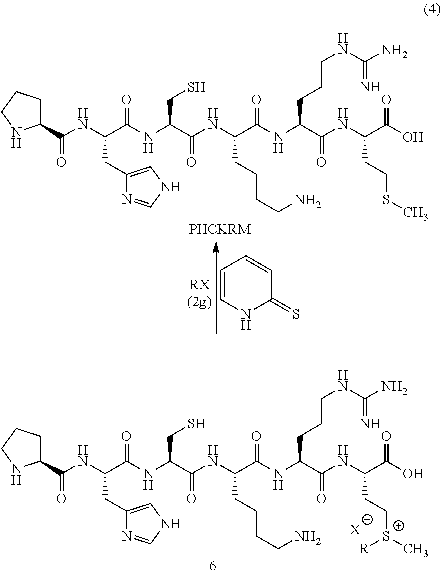

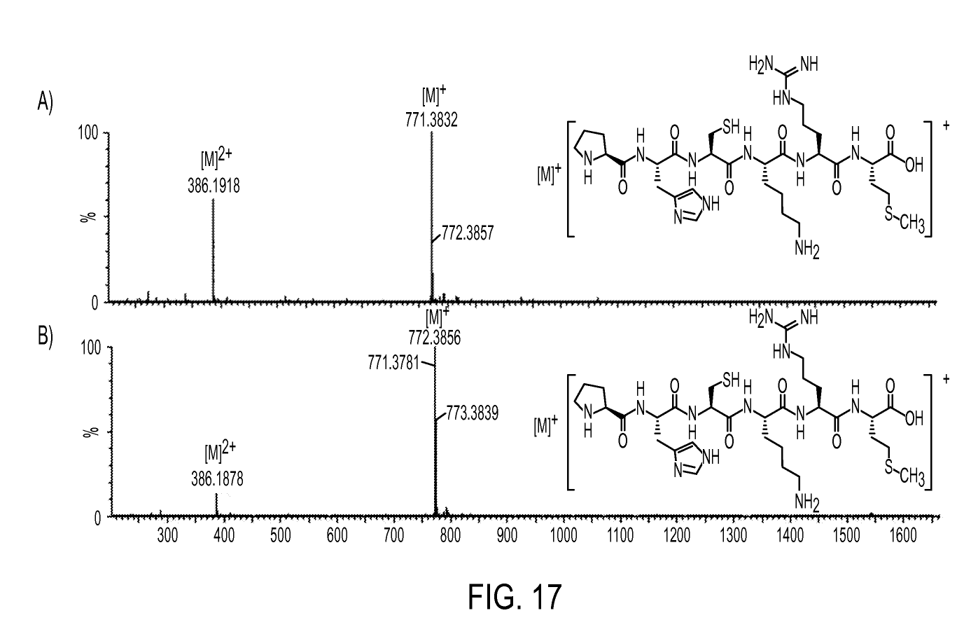

The alkylated peptide 21 was also readily dealkylated by addition of PyS to give unmodified PHCKRM as the sole product along with the alkylated PyS byproduct (Eq 2, FIGS. 10, 14, 15 and 17). This tag removal reaction is also selective, as we have found that Met sulfoniums can be dealkylated using concentrations of PyS that do not react with the disulfide bond in cystine under identical conditions (see below), which is an advantage of using PyS instead of 2-mercaptoethanol.

Overall, we have developed functionalized alkylating reagents that have been optimized for high yield, chemoselective tagging of Met residues in peptides and polypeptides. In some cases, these alkylations are completely reversible upon addition of a suitable nucleophile under mild conditions. Since the synthesis of these reagents is modular and straightforward, we envision that related compounds can be readily prepared to introduce other desirable features, such as isotopic labels to assist MS analysis (81). Once installed, the tags can be further modified using bioorthogonal reactions to introduce additional functionalities, such as affinity tags, or for attachment to substrates for purification (48, 58, 62, 72, 78, and 81). Finally, in some cases facile removal of these modified tags provides a unique advantage of this tag and modify method, where tag release may be useful for peptide concentration and purification from solid supports, as well as for release of unmodified peptide therapeutics from carriers.

### Materials and Methods

Unless stated otherwise, reactions were conducted in oven-dried glassware under an atmosphere of nitrogen using anhydrous solvents. Hexanes, THF, DCM, and DMF were purified by first purging with dry nitrogen, followed by passage through columns of activated alumina (or 4 Å molecular sieves for DMF). MeCN was freshly distilled from CaH2. Deionized water (18 MΩ-cm) was obtaining by passing in-house deionized water through a Millipore Milli-Q Biocel A10 purification unit. All commercially obtained reagents were used as received without further purification unless otherwise stated. Reaction temperatures were controlled using an IKA magnetic temperature modulator, and unless stated otherwise, reactions were performed at room temperature (RT, approximately 20° C.). Thin-layer chromatography (TLC) was conducted with EMD gel 60 F254 precoated plates (0.25 mm) and visualized using a combination of UV, anisaldehyde, and phosphomolybdic acid staining. Selecto silica gel 60 (particle size 0.032-0.063 mm) was used for flash column chromatography. ¹H NMR spectra were recorded on Bruker spectrometers (at 500 MHz) and are reported relative to deuterated solvent signals. Data for ¹H NMR spectra are reported as follows: chemical shift (δ ppm), multiplicity, coupling constant (Hz) and integration. Splitting patterns are designated as follows: s, singlet; d, doublet; t, triplet; q, quartet; m, multiplet and br, broad. ¹³C NMR spectra were recorded on Bruker spectrometers (at 125 MHz). Data for ¹³C NMR spectra are reported in terms of chemical shift. High-resolution mass spectrometry (HRMS) was performed on a Micromass Quatro-LC Electrospray spectrometer with a pump rate of 20 μL/min using electrospray ionization (ESI). Matrix assisted laser desorption ionization (MALDI) mass spectrometry was performed on an Applied Biosystems Voyager-DE STR using an α-cyano-4-hydroxycinnamic acid matrix. All Fourier Transform Infrared (FTIR) samples were prepared as thin films on NaCl plates and spectra were recorded on a Perkin Elmer RX1 FTIR spectrometer and are reported in terms of frequency of absorption (cm⁻¹). Tandem gel permeation chromatography/light scattering (GPC/LS) was performed on a 551 Accuflow Series III liquid chromatograph pump equipped with a Wyatt DAWN EOS light scattering (LS) and Optilab rEX refractive index (RI) detectors. Separations were achieved using 10⁵, 10⁴, and 10³ Å Phenomenex Phenogel 5 μm columns using 0.10 M LiBr in DMF as the eluent at 60° C. All GPC/LS samples were prepared at concentrations of 5 mg/mL.

Experimental Procedures

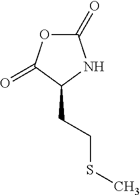

L-Methionine-N-carboxyanhydride (Met NCA), 1

To a solution of L-methionine (2.00 g, 13.4 mmol) in dry THF (0.15 M) in a Schlenk flask was added a solution of phosgene in toluene (26.8 mmol, 20% (w/v), 2 equiv.) via syringe. Caution! Phosgene is extremely hazardous and all manipulations must be performed in a well-ventilated chemical fume hood with proper personal protection and necessary precautions taken to avoid exposure. The reaction was stirred under N₂ at 50° C. for 3 hrs, then evaporated to dryness and transferred to a dinitrogen filled glove box. The condensate in the vacuum traps was treated with 50 mL of concentrated aqueous NH₄OH to neutralize residual phosgene. Crude Met NCA, a yellow oil, was purified by column chromatography¹ in 20% THF in hexanes to give 2.11 g (91%) of the product as a colorless viscous liquid that spontaneously crystallized upon standing. Spectral data was in agreement with previously published results (8).

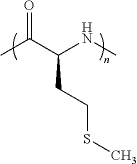

Poly(L-methionine), poly(Met), 2, General Procedure for Polymerization of Met NCA

All polymerization reactions were performed in a dinitrogen filled glove box. To a solution of Met NCA in dry THF (50 mg/mL) was rapidly added, via syringe, a solution of (PMe₃)₄Co² in dry THF (20 mM). The reaction was stirred at room temperature and polymerization progress was monitored by removing small aliquots for analysis by FTIR. Polymerization reactions were generally complete within 1 hour. Reactions were removed from the drybox and HCl (2 equiv. per (PMe₃)₄Co, 6M in H₂O) was added to the solution, which turned a blue-green color. After 10 min stirring, poly(Met) was collected by precipitation into acidic water (pH 3, HCl, >10× the reaction volume), followed by centrifugation. The white precipitate was washed with two portions of DI water and then lyophilized to yield poly(Met) as a fluffy white solid (99% yield).

¹H NMR (500 MHz, d-TFA, 25° C.): δ 5.07 (br s, 1H), 2.90 (br s, 2H), 2.48-2.29 (m, 5H).

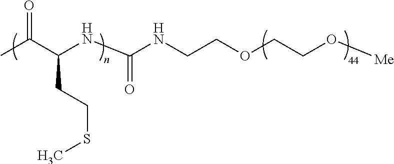

General Procedure for Endcappinq of Polymethionine with Poly(Ethylene Glycol) and Molecular Weight Determination by Endgroup Analysis (61).

The general procedure for polymerization of Met NCA was followed. Upon completion of the reaction, as confirmed by FTIR, a solution of α-methoxy-ω-isocyanoethyl-poly(ethylene glycol), PEG-NCO, (see below) in THF (3 equiv per (PMe₃)₄Co) was added to the polymerization reaction in a dinitrogen filled glove box. The reaction immediately turned from pale orange to green. The reaction was stirred overnight at room temperature and then was removed from the glove box and HCl (2 equiv per (PMe₃)₄Co, 6M in H₂O) was added to the solution, which turned a blue-green color. After 10 min stirring, endcapped poly(Met) was collected by precipitation into water (pH 3, HCl, >10× the reaction volume), followed by centrifugation. The white solids were washed with 3 portions of DI water to remove all unconjugated PEG-NCO. The PEG endcapped polymers were then isolated by lyophilization as white solids (95-99% yield). To determine poly(Met) molecular weights (Mₙ), ¹H NMR spectra were obtained. Since it has been shown that end-capping is quantitative for (PMe₃)₄Co initiated NCA polymerizations when excess isocyanate is used (61) (integrations of methionine resonances versus the polyethylene glycol resonance at δ 3.64 could be used to obtain poly(Met) lengths).

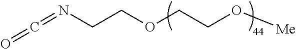

### Preparation of α-methoxy-ω-isocyanoethyl-poly(ethylene glycol)

To a solution of α-methoxy-ω-aminoethyl-poly(ethylene glycol), PEG-NH₂ (1.0 g, 0.500 mmol, Mₙ=2000 Da, Nanocs) in dry THF (25 mL) in a Schlenk flask was added a solution of phosgene in toluene (0.50 mL, 1.00 mmol, 20% (w/v) in toluene, 2 equiv) via syringe. Caution! Phosgene is extremely hazardous and all manipulations must be performed in a well-ventilated chemical fume hood with proper personal protection and necessary precautions taken to avoid exposure. The reaction was stirred under N₂ at room temperature for 16 h then evaporated to dryness and transferred to a dinitrogen filled glove box. The condensate in the vacuum traps was treated with 50 mL of concentrated aqueous NH₄OH to neutralize residual phosgene. The isocyanate was precipitated from minimal THF into 1:1 Et₂O:hexanes and was recovered as 1.01 g of a white solid (99%), no further purification.

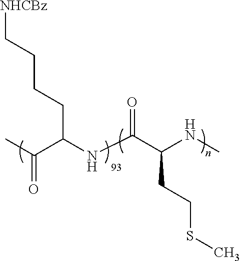

General Procedure for Polymerization of Met NCA Using Living Poly(Z-L-Lysine) Macroinitiator

Inside a dinitrogen filled glove box, a solution of Z-Lys NCA⁴ in dry THF (0.15 M) was prepared. A solution of (PMe₃)₄Co in dry THF (20 mM) was rapidly added via syringe. After 45 min, the polymerization reaction was complete as determined by FTIR. An aliquot of poly(Z-L-lysine) was removed and analyzed by GPC/LS (Mₙ=24,370, M_{w}/Mₙ=1.17, DP=93). To a solution of Met NCA in dry THF (50 mg/mL) was rapidly added via syringe, a solution of the living poly(Z-L-lysine) macroinitiator, poly(Z-lysine)₉₃, in dry THF (0.15 M). The reaction was stirred at room temperature and polymerization progress was monitored by FTIR. Polymerization reactions were generally complete within 1 hour. Reactions were removed from the drybox and HCl (2 equiv. per (PMe₃)₄Co, 6M in H₂O) was added to the solution, which then turned a blue-green color. After 10 min stirring, copolymers were collected by precipitation into acidic water (pH 3, HCl, >10× the reaction volume), followed by centrifugation. The precipitates were washed with two portions of DI water and then lyophilized to yield the poly(Z-L-lysine)₉₃-block-poly(Met)ₙ block copolymers as fluffy white solids (99% yield).

To determine polymer molecular weights by ¹H NMR spectra, all poly(Z-L-lysine)₉₃-block-poly(Met)ₙ samples were first oxidized to poly(Z-L-lysine)₉₃-block-poly(L-methionine sulfoxide)ₙ to improve their solubility.⁵ Poly(Z-L-lysine)₉₃-block-poly(Met)ₙ samples were suspended in 30% H₂O₂ in water with 1% AcOH and stirred for 30 min at 0° C. The reactions were quenched with drops of 1M sodium thiosulfate in water and then transferred to 2000 MWCO dialysis bags, and dialyzed against DI water for 48 hours with water changes twice per day. The contents of the dialysis bags were then lyophilized to dryness. Integrations of methionine resonances versus the resonances of the poly(Z-lysine)₉₃ benzyl groups found at δ 7.30 and 5.18 were used to obtain poly(Met) lengths (see example in spectral data section).

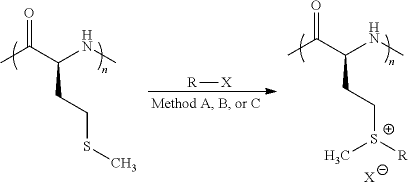

Poly(Met)Alkylation Using Activated R—X (Method A)

Poly(Met) was suspended in either DMF, water, or 0.2 M aqueous formic acid (10 mg/mL). Alkyl halide (3 eq per methionine residue) was added. 1.1 eq alkyl halide per methionine can also be used with an increased reaction time of 72 hours to give identical products. The reaction mixture was covered with foil and stirred at room temperature for 48 hours. The reaction was then diluted 2× with water, transferred to a 2000 MWCO dialysis bag, and dialyzed against 0.10 M NaCl for 24 hours, followed by DI water for 48 hours with water changes twice per day. Dialysis against NaCl serves to exchange counterions so that only chloride is present. The contents of the dialysis bag were then lyophilized to dryness to give the product as a white solid.

Poly(Met)Alkylation Using R-OTf (Method B)

Poly(Met) was dissolved in dry DCM (10 mg/mL). Alkyl triflate (2 eq per methionine residue) was added. The reaction mixture was stirred at room temperature for 48 hours. White precipitate was observed after 24 hours in all cases. After 24 hours, MeCN was added to give a 1:1 MeCN:DCM mixture to solubilize the polymer, and the resulting solution was stirred for 24 more hours. The reaction was precipitated with ether to remove excess alkyl triflate and then evaporated to dryness to give the product as a white solid. The product can then be dispersed in water, transferred to a 2000 MWCO dialysis bag, and then dialyzed against 0.10 M NaCl for 24 hours, followed by DI water for 48 hours with water changes twice per day. Dialysis against NaCl serves to exchange counterions so that only chloride is present.

Poly(Met)Alkylation Using Unactivated R—X (Method C)

Poly(Met) was suspended in dry MeCN (10 mg/mL). Alkyl halide (1.1 eq per methionine residue) was added, followed by a solution of AgBF₄ in MeCN (50 mg/mL, 1 equiv). The reaction mixture was covered with foil and stirred at 50° C. for 24 hours under N₂. A yellow precipitate was observed in all cases. The reaction was centrifuged to remove the precipitate, and polymer isolated by precipitation with ether and evaporation to dryness to give the product as a white solid. The product can then be dispersed in water, transferred to a 2000 MWCO dialysis bag, and then dialyzed against 0.10 M NaCl for 24 hours, followed by DI water for 48 hours with water changes twice per day. Dialysis against NaCl serves to exchange counterions so that only chloride is present.

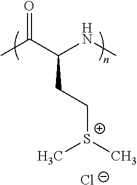

### Poly(S-methyl-L-methionine sulfonium chloride), 3

Prepared from poly(Met) and methyl iodide using method A in either DMF, water, or 0.2 M aqueous formic acid. ¹H NMR (500 MHz, D₂O, 25° C.): δ 3.46 (br s, 2H), 2.98 (br m, 6H), 2.44-2.33 (br m, 1H), 2.32-2.20 (br m, 1H).

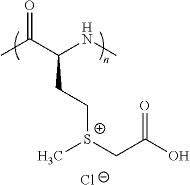

### Poly(S-carboxymethyl-L-methionine sulfonium chloride), 4

Prepared from poly(Met) and either iodoacetic acid or bromoacetic acid using method A in either DMF, water, or 0.2 M aqueous formic acid.

¹H NMR (500 MHz, D₂O, 25° C.): δ 4.56 (br s, 1H), 4.29-4.15 (br m, 2H), 3.53-3.33 (br m, 2H), 3.00-2.93 (br d, 3H), 2.37-2.33 (br m, 1H), 2.32-2.22 (br m, 1H).

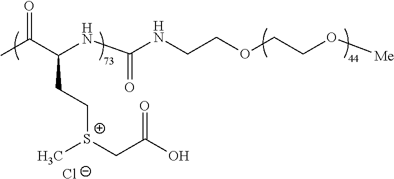

### Poly(S-carboxymethyl-L-methionine sulfonium chloride)₇₃-block-poly(ethylene glycol)₄₄

Prepared from poly(Met)₇₃-block-poly(ethyleneglycol)₄₄ and bromoacetic acid using method A in water. ¹H NMR integrals were calibrated using the polyethylene glycol resonance found at δ3.72 in D₂O. Polypeptide chain length after alkylation was in agreement with the length observed before alkylation, indicating no degradation of the polypeptide chains occurs during alkylation. ¹H NMR (500 MHz, D₂O, 25° C.): δ 4.58 (br s, 68H), 3.72 (br s, 176H), 3.52-3.36 (br m, 148H), 2.97 (br d, J=6.6, 219H), 2.46-2.35 (br m, 73H), 2.35-2.23 (br m, 73H).

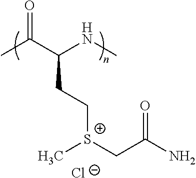

### Poly(S-carbamidomethyl-L-methionine sulfonium chloride), 5

Prepared from poly(Met) and bromoacetamide using method A in either DMF, water, or 0.2 M aqueous formic acid. ¹H NMR (500 MHz, D₂O, 25° C.): δ 4.70-4.63 (br m, 1H), 3.68-3.50 (br m, 2H), 3.12-3.06 (d, 3H), 2.53-2.43 (br m, 1H), 2.39-2.30 (br m, 1H).

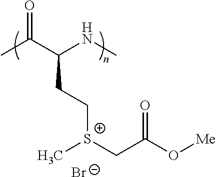

### Poly(S-(carboxymethyl methyl ester)-L-methionine sulfonium bromide), 6

Prepared from poly(Met) and methyl bromoacetate using method A in DMF. The reaction was stirred for 5 days at room temperature, then isolated by precipitation with ether. ¹H NMR (500 MHz, d-TFA, 25° C.): δ 4.85 (br s, 1H), 3.82 (s, 3H), 3.38 (br s, 2H), 2.90 (s, 3H), 2.76 (br s, 2H).

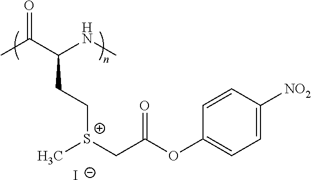

### Poly(S-(carboxymethyl p-nitrophenyl ester)-L-methionine sulfonium iodide), 7

Prepared from poly(Met) and p-nitrophenyl iodoacetate using method A. The reaction was performed in dry DMF, stirred for 5 days at room temperature, then isolated by precipitation with ether. ¹H NMR (500 MHz, d-DMSO, 25° C.): δ 8.21 (br s, 2H), 7.36 (br s, 2H), 4.35 (br s, 1H), 3.60 (br s, 2H), 2.64 (br s, 2H), 2.46 (s, 3H), 2.00-1.75 (br m, 2H).

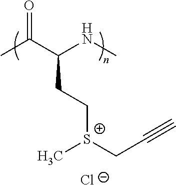

### Poly(S-propargyl-L-methionine sulfonium chloride), 8

Prepared from poly(Met) and propargyl bromide using method A in either DMF, water, or 0.2 M aqueous formic acid. ¹H NMR (500 MHz, d-TFA, 25° C.): δ 5.17 (br s, 1H), 4.66-4.45 (m 1H), 4.42-4.30 (m, 1H), 3.93-3.65 (br m, 2H), 3.12 (s, 3H), 3.01-2.55 (br m, 2H), 2.35 (s, 1H).

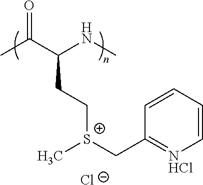

### Poly(S-(2-pyridylmethyl hydrochloride)-L-methionine sulfonium chloride), 9

Prepared from poly(Met) and 2-(bromomethyl)pyridine hydrochloride using method A in DI water. ¹H NMR (500 MHz, D₂O, 25° C.): δ 8.52 (s, 1H), 7.89 (m, 1H), 7.55 (m, 1H), 7.46 (s, 1H), 4.60 (s, 1H), 3.61-3.38 (m, 2H), 2.89 (s, 3H), 2.46-2.20 (br m, 2H).

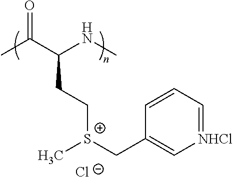

### Poly(S-(3-pyridylmethyl hydrochloride)-L-methionine sulfonium chloride), 10

Prepared from poly(Met) and 3-(bromomethyl)pyridine hydrochloride using method A in DI water. ¹H NMR (500 MHz, D₂O, 25° C.): δ 8.59 (s, 2H), 7.97 (s, 1H), 7.53 (s, 1H), 4.54 (s, 1H), 3.53-3.01 (m, 2H), 2.87 (s, 3H), 2.45-2.18 (br m, 2H).

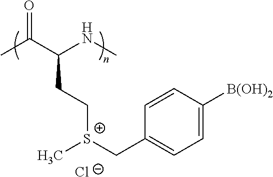

### Poly(S-(4-boroxyphenylmethyl)-L-methionine sulfonium chloride), 11

Prepared from poly(Met) and 4-(bromomethyl)phenylboronic acid using method A in DMF. ¹H NMR (500 MHz, D₂O, 25° C.): δ 7.68 (br s, 2H), 7.37 (br s, 2H), 4.71-4.60 (br m, 1H), 4.55 (br s, 2H), 3.45-3.22 (br m, 2H), 2.82-2.71 (br m, 3H), 2.38-2.12 (br m, 2H).

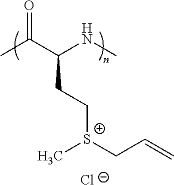

### Poly(S-allyl-L-methionine sulfonium chloride), 12

Prepared from poly(Met) and allyl iodide (prepared from commercially available allyl chloride using the Finkelstein reaction (48)) using method C. ¹H NMR (500 MHz, d-TFA, 25° C.): δ 5.94-5.86 (br m, 1H), 5.81-5.71 (br m, 2H), 4.93 (br s, 1H), 4.18-4.11 (br m, 1H), 4.01-3.95 (br m, 1H), 3.60-3.41 (br m, 2H), 2.89 (br s, 3H), 2.63 (br s, 1H), 2.52-2.42 (br s, 1H).

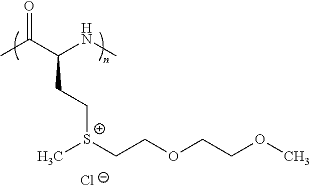

### Poly(S-(2-(2-methoxyethoxy)ethyl)-L-methionine sulfonium chloride), 13

Prepared from poly(Met) and 1-iodo-2-(2-methoxyethoxy)ethane using method C. After reaction completion and removal of silver iodide by centrifugation, the polymer was precipitated from solution with ether and collected by centrifugation. The white solids where taken up in water and transferred to a 2000 MWCO dialysis bag, and dialyzed against 0.10 M NaCl for 24 hours, followed by DI water for 48 hours with water changes twice per day. The contents of the dialysis bag were then lyophilized to dryness to give the product as a white solid. ¹H NMR (500 MHz, d-TFA, 25° C.): δ 4.67 (br s, 1H), 3.95 (br s, 1H), 3.84-3.56 (br m, 9H), 3.48 (br s, 3H), 2.88 (s, 3H), 2.52 (br s, 1H), 2.35 (br s, 1H).

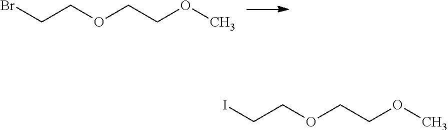

1-Iodo-2-(2-methoxyethoxy)ethane was prepared from commercial 1-bromo-2-(2-methoxyethoxy)ethane using the Finkelstein reaction. 1-Bromo-2-(2-methoxyethoxy)ethane (0.100 g, 0.546 mmol, 1 eq) was dissolved in acetone (5 mL, dried over MgSO₄) and sodium iodide (0.246 g, 1.64 mmol, 3 eq) was added. The reaction was covered with foil and stirred at 40° C. for 16 hours under N₂. The reaction was evaporated to dryness by rotary evaporation. The residue was taken up into 75 mL EtOAc and washed with 25 mL of 0.1 M sodium thiosulfate, and 2×50 mL of brine. The organic phase was dried over MgSO₄ and condensed by rotary evaporation to give 0.125 g of 1-iodo-2-(2-methoxyethoxy)ethane as a pale yellow oil (99%). ¹H NMR (500 MHz, CDCl₃, 25° C.): δ 3.73 (t, J=7.0, 2H), 3.63-3.62 (m, 2H), 3.54-3.52 (m, 2H), 3.36 (s, 3H), 3.24 (t, J=7.0, 2H); ¹³C NMR (125 MHz, CDCl₃, 25° C.): δ71.9, 71.8, 70.0, 59.0, 2.6. HRMS-ESI m/z) [M+H]⁺ Calculated for C₅H₁₂IO₂, 230.98. found 230.98.

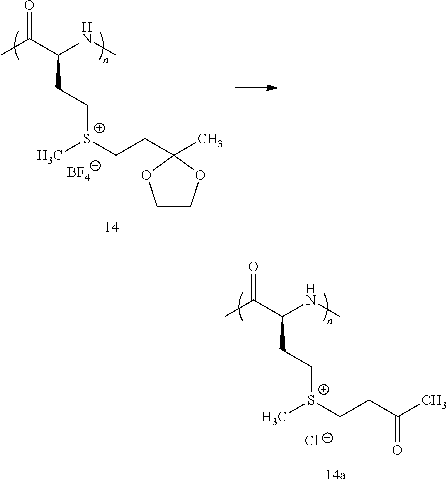

### Poly(S-(3-oxybutyl)-L-methionine sulfonium chloride), 14a

Prepared from poly(Met) and bromoethyl-2-methyl-1,3-dioxolane using method C. After reaction completion and removal of silver iodide by centrifugation, the polymer was precipitated from solution with ether and collected by centrifugation (99% yield). Deprotection to give the ketone groups was accomplished during dialysis (2000 MWCO tubing) against 2M HCl with 0.10 M NaCl for 24 hours, followed by DI water for 48 hours with water changes twice per day. The contents of the dialysis bag were then lyophilized to dryness to give the product 14a as a white solid. (98%) ¹H NMR (500 MHz, D₂O, 25° C.): δ 4.63-4.44 (br m, 1H), 3.65-3.38 (br m, 4H), 3.27 (br s, 2H), 2.99 (s, 3H), 2.64 (br s, 1H), 2.41 (br s, 1H), 2.28 (s, 3H). ¹⁹F NMR (400 MHz, D₂O, 25° C.): no signals.

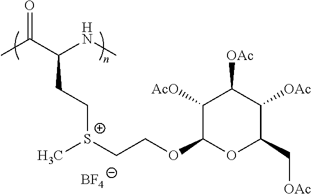

### Poly(S-2-(2,3,4,6-tetra-O-acetyl-α-D-glucopyranosyl)oxyethyl-L-methionine sulfonium tetrafluoroborate), 15

Prepared from poly(Met) and 1-iodo-2-(2,3,4,6-tetra-O-acetyl-α-D-glucopyranosyl)ethane using method C. ¹H NMR (500 MHz, d-TFA, 25° C.): δ 5.60-5.53 (br m, 1H), 5.44-5.26 (br m, 1H), 4.86 (br s, 1H), 4.46 (br s, 3H), 4.24 (br s, 1H), 4.03 (br s, 1H), 3.90 (br s, 1H), 3.58 (br s, 2H), 3.39 (br s, 2H), 3.03 (d, J=21.5, 3H), 2.59 (br s, 1H), 2.37-2.13 (br m, 13H). ¹⁹F NMR (400 MHz, d-DMAC, 25° C.): δ −79.5.

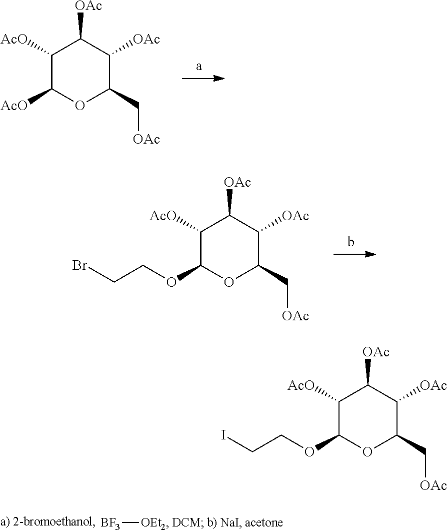

### Preparation of 1-iodo-2-(2,3,4,6-tetra-O-acetyl-α-D-glucopyranosyl)ethane

1-Bromo-2-(2,3,4,6-tetra-O-acetyl-α-D-glucopyranosyl)ethane was prepared from commercial glucose pentaacetate according to a literature method⁷, and then converted to 1-iodo-2-(2,3,4,6-tetra-O-acetyl-α-D-glucopyranosyl)ethane using the Finkelstein reaction. 1-Bromo-2-(2,3,4,6-tetra-O-acetyl-α-D-glucopyranosyl)ethane (1.17 g, 2.56 mmol, 1 eq) was dissolved in acetone (30 mL, dried over MgSO₄) and sodium iodide (1.15 g, 7.68 mmol, 3 eq) was added. The reaction was covered with foil and stirred at 40° C. for 16 hours under N₂. The reaction was evaporated to dryness by rotary evaporation. The residue was taken up into 75 mL EtOAc and washed with 25 mL of 0.1 M sodium thiosulfate, 50 mL of water, and 50 mL of brine. The organic phase was dried over MgSO₄ and condensed by rotary evaporation to give 1.26 g of 1-iodo-2-(2,3,4,6-tetra-O-acetyl-α-D-glucopyranosyl)ethane (98%).

¹H NMR (500 MHz, CDCl₃, 25° C.): δ 5.06 (dd, J=9.5, 9.5, 1H), 4.92 (dd, J=9.6, 9.6, 1H), 4.88 (dd, J=8.7, 8.7, 1H), 4.46 (d, J=7.9, 1H), 4.11 (dd, J=12.0, 4.9, 1H), 4.00-3.92 (m, 2H), 3.67-3.52 (m, 2H), 3.19-3.06 (m, 2H), 1.93 (s, 3H), 1.92 (s, 3H), 1.87 (s, 3H), 1.85 (s, 3H); ¹³C NMR (125 MHz, CDCl₃, 25° C.): 6170.2, 169.8, 169.2, 169.0, 100.4, 72.4, 71.6, 70.8, 70.2, 68.1, 20.6, 20.5, 20.4, 2.2; HRMS-ESI m/z) [M+H]⁺ C₁₆H₂₄IO₁₀,502.03. found 502.03.

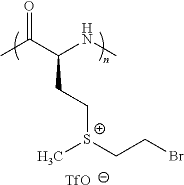

### Poly(S-(2-bromoethyl)-L-methionine sulfonium triflate), 16

Prepared from poly(Met) and 2-bromoethyltriflate (prepared from commercially available 2-bromoethanol) using method B. ¹H NMR (500 MHz, d-TFA, 25° C.): δ 4.89 (br s, 1H), 4.06-3.95 (br m, 1H), 3.89-3.53 (br m, 5H), 3.07 (br s, 3H), 2.61 (br s, 1H), 2.43 (br s, 1H). ¹⁹F NMR (400 MHz, d-DMAC, 25° C.): δ −79.3.

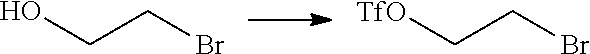

2-Bromoethyltriflate was prepared using a modified literature procedure (8). 2-Bromoethanol (0.5 g, 4.00 mmol, 0.284 mL, 1 eq) was dissolved in dry DCM (15 mL) and dry pyridine was added (0.380 g, 4.80 mmol, 0.387 mL, 1.2 eq) and cooled to 0° C. under N₂. Triflic anhydride (1.24 g, 4.40 mmol, 0.740 mL, 1.1 eq, previously distilled over P₂O₅) was added dropwise and the reaction stirred for 20 min. The reaction was diluted with 100 mL of EtOAc and washed with 2×50 mL of water at pH 3 (HCl) to remove pyridine and pyridine salts, followed by 50 mL of 10% aqueous bicarbonate, and finally 25 mL of brine. The organic phase was dried over MgSO₄ and condensed by rotary evaporation at 25° C. to give 1.02 g of 2-bromoethyltriflate as a clear oil (99%), which was stored at −20° C. under N₂ and used with no further purification. Spectral data was in agreement with previously published results.⁸

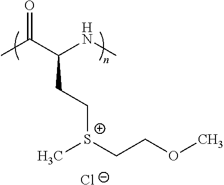

### Poly(S-(2-methoxyethyl)-L-methionine sulfonium chloride), 17

Prepared from poly(Met) and 2-methoxyethyl triflate (prepared from commercially available 2-methoxyethanol) using method B. Upon reaction completion, polymer was precipitated from solution with ether and collected by centrifugation. The white solids where taken up in water and transferred to a 2000 MWCO dialysis bag, and dialyzed against 0.10 M NaCl for 24 hours, followed by DI water for 48 hours with water changes twice per day. The contents of the dialysis bag were then lyophilized to dryness to give the product as a white solid. ¹H NMR (500 MHz, D₂O, 25° C.): δ 4.58 (br s, 1H), 3.94 (br s, 2H), 3.74-3.35 (br m, 7H), 3.01 (d, J=5.7, 3H), 2.38 (br s, 1H), 2.26 (br s, 1H). ¹⁹F NMR (400 MHz, D₂O, 25° C.): no signals.

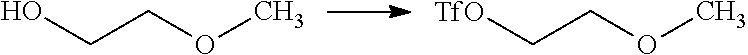

2-methoxyethanol (0.500 g, 6.57 mmol, 0.518 mL, 1 eq.) was dissolved in dry DCM (15 mL) and dry pyridine was added (0.623 g, 7.88 mmol, 0.635 mL, 1.2 eq.) and the mixture cooled to 0° C. under N₂. Triflic anhydride (2.04 g, 7.23 mmol, 1.22 mL, 1.1 eq., previously distilled over P₂O₅) was added dropwise and the reaction stirred for 20 min. The reaction was diluted with 100 mL of EtOAc and washed with 2×50 mL 1 M NaCl at pH 3 (HCl) to remove pyridine and pyridine salts, followed by 25 mL of brine. The organic phase was dried over MgSO₄ and condensed by rotary evaporation at 25° C. to give 1.33 g of 2-methoxyethyl triflate as a clear oil (97%). The triflate was used directly with no further purification.

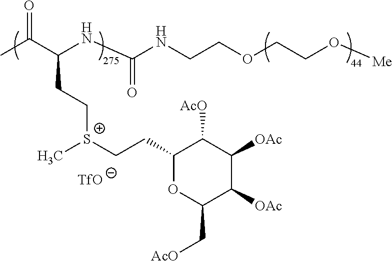

### Poly(S-2-(2,3,4,6-tetra-O-acetyl-α-D-galactopyranosyl)ethyl-L-methionine sulfonium triflate)-block-PEG₄₄, 18

Prepared from poly(Met)-block-poly(ethylene glycol)₄₄ and 2-(2,3,4,6-tetra-O-acetyl-α-D-galactopyranosyl)ethyl triflate using method B. ¹H NMR integrals were calibrated using the polyethylene glycol resonance found at δ3.72 in D₂O. Polypeptide chain length after alkylation was in agreement with the length observed before alkylation. ¹H NMR (500 MHz, d-TFA, 25° C.): δ 5.65 (br s, 282H), 5.45-5.41 (br m, 553H), 4.85 (br m, 567H), 4.62-4.28 (br m, 1124H), 3.78 (s, 176H), 3.75-3.40 (br m, 1110H), 3.07 (d, J=23.5 840H), 2.67 (br s, 548H), 2.43 (br s, 544H), 2.27-2.18 (br m, 3260H). ¹⁹F NMR (400 MHz, d-DMAC, 25° C.): δ −79.1.

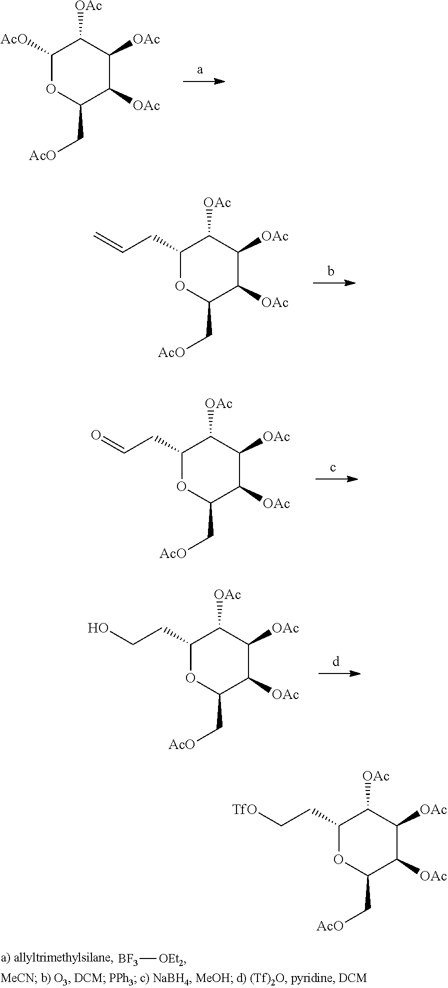

### Preparation of 2-(2,3,4,6-tetra-O-acetyl-α-D-galactopyranosyl)ethyl triflate

2-(2,3,4,6-tetra-O-acetyl-α-D-galactopyranosyl)ethanol was prepared using previously published procedures via allylation of galactose pentaacetate (58, and 62), followed by ozonolysis (72), and reduction of the aldehyde (45). 2-(2,3,4,6-Tetra-O-acetyl-α-D-galactopyranosyl)ethanol (20.5 mg, 0.054 mmol, 1 eq) was dissolved in dry DCM (1.5 mL), dry pyridine was added (5.2 mg, 0.065 mmol, 5.3 μL, 1.2 eq.), and the mixture cooled to 0° C. under N₂. Triflic anhydride (16.9 mg, 0.060 mmol, 10.1 μL, 1.1 eq., previously distilled over P₂O₅) was added and the reaction stirred for 20 min. The reaction was diluted with 50 mL of EtOAc and washed with 2×20 mL of water at pH 3 (HCl) to remove pyridine and pyridine salts, followed by 20 mL of 10% aqueous bicarbonate, and finally 20 mL of brine. The organic phase was dried over MgSO₄ and condensed by rotary evaporation at 25° C. to give 2-(2,3,4,6-tetra-O-acetyl-α-D-galactopyranosyl)ethyl triflate as a clear oil (28 mg, >99% yield) which was used directly with no further purification.

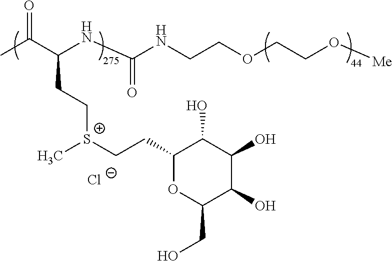

### Poly(S-2-(α-D-galactopyranosyl)ethyl-L-methionine sulfonium chloride)-block-poly(ethylene glycol)₄₄

To a solution of poly(S-2-(2,3,4,6-tetra-O-acetyl-α-D-galactopyranosyl)ethyl-L-methionine sulfonium triflate)-block-poly(ethylene glycol)₄₄ in methanol (10 mg/mL) was added hydrazine monohydrate (2 equiv. per OAc group) and the reaction stirred overnight at room temperature. The reaction was quenched by addition of a few drops of acetone. Et₂O was added and the white solids were collected by centrifugation, (99% yield). To exchange counterions, the solids were taken up with water and transferred to 2000 molecular weight cutoff dialysis tubing and dialyzed against 0.10 M NaCl for 24 hours, followed by DI water for 48 hours with water changes twice per day. The contents of the dialysis bag were then lyophilized to dryness to give the product as a white solid (88% yield). ¹H NMR (500 MHz, D₂O, 25° C.): δ 4.58-4.41 (br m, 640H), 4.16 (br s, 275H), 4.02-3.96 (br m, 287H), 3.94 (br s, 270H), 3.77-3.70 (br m, 870H), 3.66 (s, 177H), 3.53-3.30 (br m, 1095H), 2.96 (br s, 817H), 2.63-2.47 (br m, 580H), 2.41-1.96 (br m, 617H). ¹⁹F NMR (400 MHz, D₂O, 25° C.): no signals.

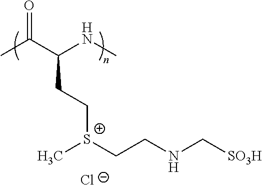

### Poly-S-(4-sulfonato-3-azabutyl)-L-methionine sulfonium chloride, 19

A solution of amino methane sulfonic acid (3 eq.) in saturated NaHCO₃ (25 mg/ml, pH 10) was added to a solution of poly-S-2-bromoethyl-L-methionine sulfonium triflate in MeCN (20 mg/mL). The reaction was stirred overnight at room temperature, then transferred to a 2000 MWCO dialysis bag, and dialyzed against 0.10 M NaCl for 24 hours, followed by DI water for 48 hours with water changes twice per day. The contents of the dialysis bag were then lyophilized to dryness to give a white solid. ¹H NMR (500 MHz, D₂O, 25° C.): δ4.60 (s, 1H), 3.74 (m, 2H), 3.55-3.40 (m, 6H), 3.04 (s, 3H), 2.48-2.24 (m, 2H). ¹⁹F NMR (400 MHz, D₂O, 25° C.): no signals.

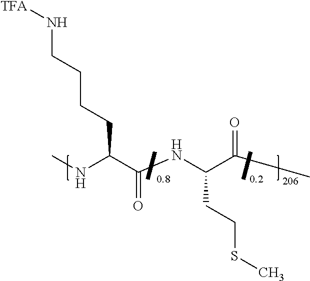

### Poly[(Nε-L-TFA-lysine)_{0.8}-stat-(L-Methionine)_{0.2}]₂₀₆, 20

Inside a dinitrogen filled glove box, a solution of N_{ε}-TFA-L-lysine-N-carboxyanhydride, TFA-Lys NCA, (440 mg, 1.64 mmol, 8 eq) and Met NCA (71.7 mg, 0.409 mmol, 2 eq) in dry THF (0.15 M) was prepared. A solution of (PMe₃)₄Co in dry THF (20 mM) was rapidly added via syringe (18.6 mg, 0.051 mmol, 0.025 eq). After 45 min, the polymerization reaction was complete as determined by FTIR. An aliquot was removed and analyzed by GPC/LS (Mₙ=46,180, PDI=1.07, DP=206). The polymerization was removed from the drybox and HCl (2 eq per (PMe₃)₄Co, 6M in H₂O) was added to the solution, which turned a blue-green color. After 10 min stirring, the copolymer was collected by precipitation into acidic water (pH 3, HCl, >10× the reaction volume), followed by centrifugation. The white precipitate was washed with two portions of DI water and then lyophilized to give the copolymer as a fluffy white solid (99% yield). Polypeptides used in this study were either 32 (Mₙ=6,520, M_{w}/Mₙ=1.21) or 135 (Mₙ=27,730, M_{w}/Mₙ=1.21) residues in length as determined by GPC analysis, and both gave similar results. ¹H NMR (500 MHz, d-TFA, 25° C.): δ 4.63 (br s, 1H), 3.48 (br s, 2H), 2.46-1.37 (br m, 9H). ¹⁹F NMR (400 MHz, MeOD, 25° C.): −75.3.

General Procedure for Preparation of Poly[(N_{ε}-trifluoroacetyl-L-lysine)_{0.8}-stat-(L-methionine)_{0.2}]ₙ

Inside a dinitrogen filled glove box, a solution of N_{ε}-TFA-L-lysine-N-carboxyanhydride (42) (TFA-Lys NCA), (616 mg, 2.30 mmol, 4 eq) and L-methionine-N-carboxyanhydride (Met NCA) (41) (100 mg, 0.573 mmol, 1 eq) in dry THF (0.15 M) was prepared. A solution of (PMe₃)₄Co (39) in dry THF (20 mM) was rapidly added via syringe (26.0 mg, 0.0714 mmol, 0.025 eq). After 45 min, the polymerization reaction was complete as determined by FTIR. An aliquot was removed and analyzed by GPC/LS. The polymerization was removed from the drybox and HCl (2 eq. per (PMe₃)₄Co, 6M in H₂O) was added to the solution, which turned a blue-green color. After 10 min stirring, the copolymer was collected by precipitation into acidic water (pH 3, HCl, >10× the reaction volume), followed by centrifugation. The white precipitate was washed with two portions of DI water and then lyophilized to give the copolymer as a fluffy white solid (595 mg, 99% yield). Spectral data was in agreement with previous reports (42). Polypeptides used in this study were either 32 (Mₙ=6,520, M_{w}/Mₙ=1.21) or 135 (Mₙ=27,730, M_{w}/Mₙ=1.21) residues in length as determined by GPC analysis, and both gave similar results.

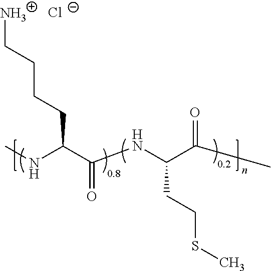

General Procedure for Deprotection of TFA-Lysine Poly[(L-lysine.HCl)_{0.8}-stat-(L-methionine)_{0.1}]ₙ, KM, 1

Poly[(N_{ε}-trifluoroacetyl-L-lysine)_{0.8}-stat-L-methionine)_{0.2}]ₙ was dispersed in methanol:water, 9:1 (20 mg/mL) and K₂CO₃ (2 eq per lysine residue) was added. The reaction was stirred for 8 hours at 50° C. and then the methanol was removed by rotary evaporation. The remaining solution was acidified to pH 3 with 6M HCl, and transferred to 2000 MWCO dialysis tubing. The polypeptide was dialyzed at pH 4 (HCl) for 24 hours, followed by DI water for 48 hours with water changes twice per day. The contents of the dialysis tubing were then lyophilized to dryness to give poly[(L-lysine.HCl)_{0.8}-stat-(L-methionine)_{0.2}]ₙ, KM as a white solid. (82% yield) ¹H NMR (500 MHz, D₂O, 25° C.): δ 4.51 (s, 1H), 4.32 (s, 4H), 3.02 (m, 8H), 2.66-2.52 (m, 2H), 2.16-1.97 (m, 5H), 1.88-1.66 (m, 16H), 1.46 (s, 8H).

General Procedure for Alkylation of KM

KM was dissolved in 0.2 M formic acid (10 mg/mL) and the alkylating reagent (1.5 eq per methionine residue) was added. The reaction mixture was covered with foil and stirred at room temperature for 48 hours. The reaction was then transferred to 2000 MWCO dialysis tubing and dialyzed against 0.10 M NaCl for 24 hours to exchange all counterions to chloride, followed by dialysis against DI water for 48 hours with water changes twice per day. The contents of the dialysis tubing were then lyophilized to dryness to give the product as a white solid. Note: Reactions performed with 1.0 eq of alkylating reagent completed with reaction times extended to 64 hours.

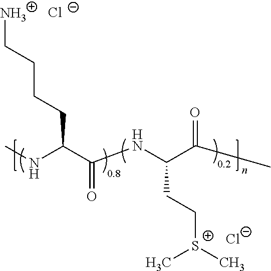

### Poly[(L-lysine.HCl)_{0.8}-stat-(S-methyl-L-methionine sulfonium chloride)_{0.2}]ₙ, 3a

Polysulfonium 3a was prepared from KM and methyl iodide according to the general procedure for alkylation of KM, (88% yield). ¹H NMR (500 MHz, D₂O, 25° C.): δ ¹H NMR (500 MHz, D₂O, 25° C.): δ 4.56 (s, 1H), 4.28 (s, 4H), 3.39 (s, 2H), 3.04-2.89 (m, 12H), 2.37-2.16 (m, 2H), 1.84-1.60 (m, 16H), 1.45 (s, 8H).

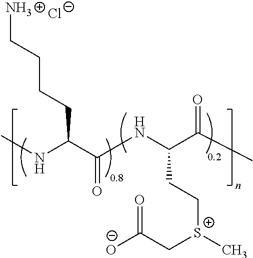

### Poly[(L-lysine.HCl)_{0.8}-stat-(S-(carboxymethyl)-L-methionine)_{0.2}]ₙ, 3b

Polysulfonium 3b was prepared from KM and bromoacetic acid according to the general procedure for alkylation of KM, (85% yield). ¹H NMR (500 MHz, D₂O, 25° C.): δ ¹H NMR (500 MHz, D₂O, 25° C.): δ 4.56 (s, 1H), 4.26 (s, 4H), 3.46-3.28 (m, 2H), 3.04-2.84 (m, 12H), 2.37-2.14 (m, 2H), 1.85-1.59 (m, 16H), 1.42 (s, 8H).

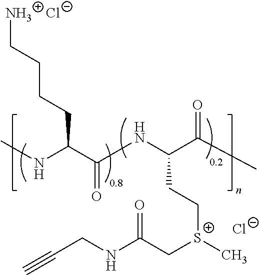

### Poly[(L-lysine-HCl)_{0.8}-stat-(S—(N-propargyl-acetamido)-L-methionine sulfonium chloride)_{0.2}]ₙ, 3c

Polysulfonium 3c was prepared from KM and N-propargyl-bromoacetamide 2c according to the general procedure for alkylation of KM, (85% yield). ¹H NMR (500 MHz, D₂O, 25° C.): δ 4.62 (s, 1H), 4.32 (s, 4H), 4.06 (s, 2H), 3.64-3.46 (m, 2H), 3.07-2.97 (m, 12H), 2.69 (s, 1H), 2.44-2.20 (m, 2H), 1.88-1.62 (m, 16H), 1.47 (s, 8H).

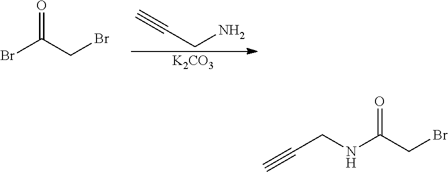

### Preparation of N-propargyl-bromoacetamide, 2c

N-propargyl-bromoacetamide was prepared from bromoacetyl bromide according to a modified literature procedure (70). Propargyl amine (0.166 mL, 2.60 mmol, 1.05 eq) was added dropwise to a solution of K₂CO₃ (0.358 g, 2.60 mmol, 1.05 eq) and bromoacetyl bromide (0.500 g, 2.48 mmol, 1.00 eq) in CH₂Cl₂ (20 mL) at 0° C. The resulting solution was allowed to reach RT and stir for 4 hours. The reaction was filtered, the filter cake rinsed with CH₂Cl₂, and the filtrate was evaporated to a brown solid, which was recrystallized from THF and hexanes to give N-propargyl-bromoacetamide (0.313 g, 72%). Spectral data were consistent with literature values.

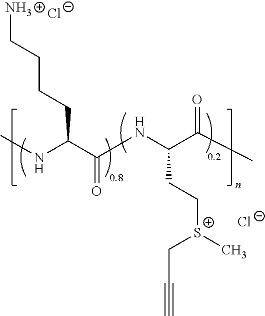

### Poly[(L-lysine.HCl)_{0.8}-stat-(S-propargyl-L-methionine sulfonium chloride)_{0.2}]ₙ, 3d

Polysulfonium 3d was prepared from KM and propargyl bromide according to the general procedure for alkylation of KM, (92% yield). ¹H NMR (500 MHz, 2% d-TFA in D₂O, 25° C.): δ 4.44 (s, 1H), 4.15 (s, 6H), 3.31 (s, 2H), 2.89-2.82 (m, 16H), 2.26-2.15 (m, 3H), 2.08 (s, 1H), 1.70-1.48 (m, 265H), 1.30 (s, 13H).

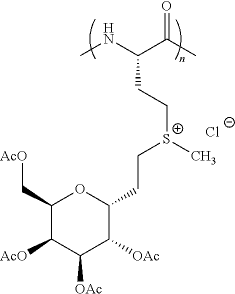

### Poly(S-2-(2,3,4,6-tetra-O-acetyl-α-D-galactopyranosyl)ethyl-L-methionine sulfonium chloride), 15

Polysulfonium 15 was prepared as previously described (42).

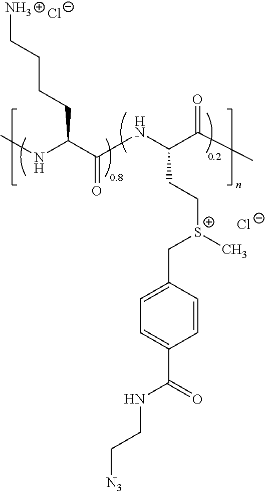

### Poly[(L-lysine.HCl)_{0.8}-stat-(S-4-(N-azidoethylcarboxamide)phenylmethyl-L-methionine sulfonium chloride)_{0.2}]ₙ, 3f

Polysulfonium 3f was prepared from KM and α-bromomethyl-(N-azidoethyl)-p-toluamide 2f according to the general procedure for alkylation of KM, except that α-bromomethyl-(N-azidoethyl)-p-toluamide was added as a 25 mg/mL solution in ethanol (87% yield). ¹H NMR (500 MHz, D₂O, 25° C.): δ 7.91-7.307 (m, 4H), 4.47 (s, 1H), 4.19 (s, 3H), 3.58-3.11 (m, 4H), 2.89 (s, 1H), 2.74 (s, 1H), 2.25-1.96 (m, 2H), 1.78-1.11 (m, 23H).

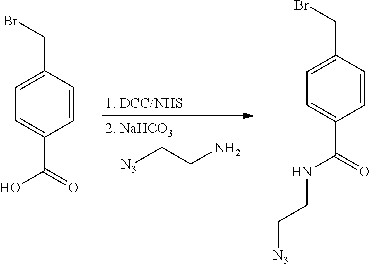 [SMILES: O=C(O)c1ccc(CBr)cc1 ; [N-]=[N+]=NCCN ; [N-]=[N+]=NCCNC(=O)c1ccc(CBr)cc1]

### Preparation of α-bromomethyl-(N-azidoethyl)-p-toluamide, 2f

The NHS ester of α-bromomethyl-p-toluic acid was prepared according to a literature procedure (Jacobsen, K. A.; Furlano, D. C.; Kirk, K. L. J. Fluorine Chem. 1988, 39, 339-347). α-Bromomethyl toluic acid (0.140 g, 0.651 mmol, 1.00 eq) was dissolved in DMF/ethyl acetate 1/1 (5 mL). NHS (0.0787 g, 0.684 mmol, 1.05 eq) and then DCC (0.141 g, 0.684 mmol, 1.05 eq) were added. A white precipitate formed within 10 minutes. The reaction was stirred for 2 hours, filtered, the filter cake was washed with ethyl acetate, and the filtrate was condensed to a white solid. The crude NHS ester was redissolved in DMF (5 mL) and K₂CO₃ was added (0.105 g, 0.716 mmol, 1.10 eq) followed by 2-azidoethylamine (1) (0.0654 g, 0.716 mmol, 1.10 eq). The reaction was stirred for 4 hours, then diluted with water (200 mL). The product was extracted with 3 portions of ethyl acetate (50 mL), the combined organic layers were washed with water and brine, dried over sodium sulfate, and condensed. The pale yellow solid was chromatographed on silica in 5% methanol in benzene to give α-bromomethyl-(N-azidoethyl)-p-toluamide, 0.140 g (76%). ¹H NMR (500 MHz, CDCl₃, 25° C.): δ 7.74 (d, ³J (H, H)=8.0, 2H), 7.43 (d, ³J (H, H)=8.0, 2H), 6.70 (s, 1H), 4.49 (s, 2H), 3.61-3.58 (m, 2H), 3.53 (t, ³J (H, H)=5.5, 2H). ¹³C NMR (125 MHz, CDCl₃, 25° C.): δ 167.0, 141.3, 133.9, 129.2, 127.4, 50.7, 39.4, 32.2. HRMS-ESI (m/z) [M+H]⁺ C₁₀H₁₁BrN₄O, calcd: 282.01. found: 282.01.

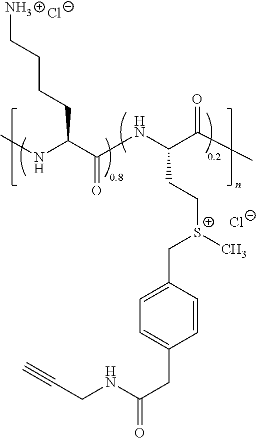

### Poly[(L-lysine.HCl)_{0.8}-stat-(S-(4-(N-propargyl-acetamido)phenylmethyl)-L-methionine sulfonium chloride)_{0.2}]ₙ, 3g

Polysulfonium 3g was prepared from KM and 4-bromomethyl-N-propargyl-phenylacetamide 2g according to the general procedure for alkylation of KM, except that 4-bromomethyl-N-propargyl-phenylacetamide was added as a 25 mg/mL solution in ethanol, (92% yield). ¹H NMR (500 MHz, D₂O, 25° C.): δ 7.39-7.24 (m, 4H), 4.48 (s, 1H), 4.19 (s, 4.5H), 3.85 (s, 2H), 3.57-3.48 (m, 2H), 3.34-3.13 (m, 2H), 2.90 (m, 9H), 2.73-2.61 (m, 3H), 2.50 (s, 1H), 2.23-2.03 (m, 2H), 1.74-1.48 (m, 18H), 1.34 (s, 9H).

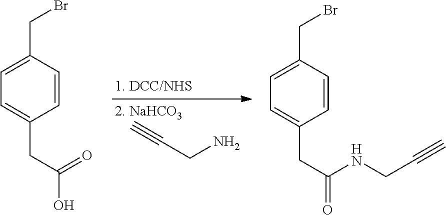

### Preparation of 4-bromomethyl-N-propargyl-phenylacetamide, 2q

4-Bromomethyl-phenylacetic acid (0.509 g, 2.22 mmol, 1.00 eq) was dissolved in dry THF (20 mL). NHS (0.258 g, 2.24 mmol, 1.01 eq) and then DCC (0.463 g, 2.24 mmol, 1.01 eq) were added. A white precipitate formed within 10 minutes. The reaction was stirred for 2 hours, filtered, the filter cake was washed with THF, and the filtrate was condensed to a white solid. The crude NHS ester was redissolved in DMF (20 mL) and K₂CO₃ was added (0.307 g, 2.22 mmol, 1.00 eq) followed by propargyl amine (0.149 mL, 2.33 mmol, 1.05 eq). The reaction was stirred for 4 hours, then diluted with water (200 mL). The product was extracted with 3 portions of ethyl acetate (50 mL), the combined organic layers were washed with water and brine, dried over sodium sulfate, and condensed. The pale yellow solid was chromatographed on silica in 5% methanol in benzene to give 4-bromomethyl-N-propargyl-phenylacetamide, 0.496 g (84%). ¹H NMR (500 MHz, MeOD, 25° C.): δ 7.38 (d, ³J (H, H)=8.2, 2H), 7.28 (d, ³J (H, H)=8.2, 2H), 4.55 (s, 2H), 3.95 (d, ³J (H, H)=2.5, 2H), 3.51 (s, 2H), 2.58 (t, ³J (H, H)=2.6, 1H). ¹³C NMR (125 MHz, CDCl₃, 25° C.): δ 170.0, 137.0, 134.5, 129.8, 129.6, 79.2, 71.6, 43.0, 32.9, 29.3. HRMS-ESI (m/z) [M+H]⁺ C₁₂H₁₃BrNO, calculated for 266.01. found 266.02.

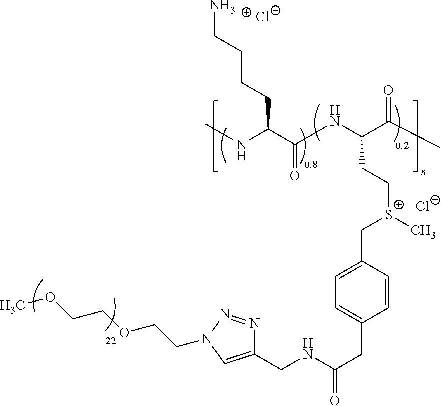

### Copper(I)-catalyzed Azide-Alkyne Cycloaddition of azide terminated polyethyleneglycol with sulfonium 3g to give 5a

Polysulfonium 3g was dissolved in water (5 mg/mL) and azide terminated polyethyleneglycol (Sigma, MW=1,000), (1.2 eq/alkyne) was added. The solution was degassed by bubbling N₂ through the solution for 20 minutes and then stirred under N₂. Separately, a solution of Cu(I) was prepared by addition of sodium ascorbate (0.5 eq/alkyne) to a degassed solution of Cu(II)SO₄ (0.1 eq/alkyne) and pentamethyldiethylenetriamine (0.1 eq/alkyne). The solution turned dark blue. The Cu(I) solution was transferred to the azide/alkyne solution via syringe. The reaction was stirred at room temperature for 48 hours and then transferred to 8000 MWCO dialysis tubing and dialyzed against 0.10 M NaCl for 24 hours, followed by dialysis against DI water for 72 hours with water changes twice per day. The contents of the dialysis tubing were then lyophilized to dryness to give the product, 5a, as a white solid (95% yield). ¹H NMR (500 MHz, D₂O, 25° C.): δ 7.92 (s, 1H), 7.46-7.18 (m, 4H), 4.27 (s, 4H), 4.00-3.42 (m, 152H), 3.20 (s, 2H), 2.97 (s, 8H), 2.77 (s, 3H), 2.53 (m, 2H), 2.19 (m, 2H), 1.83-1.59 (m, 16H), 1.42 (s, 8H).

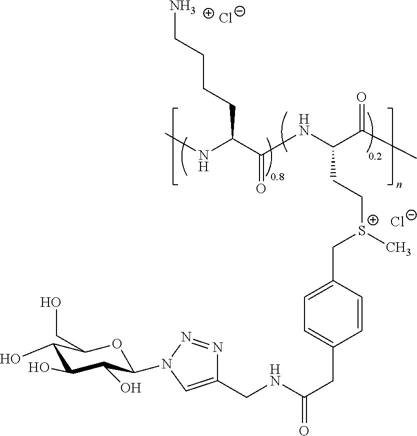

### Copper(I)-catalyzed Azide-Alkyne Cycloaddition of 6-D-glucopyranosyl azide with sulfonium 3g to give 5b

Polysulfonium 3g was dissolved in water (5 mg/mL) and β-D-glucopyranosyl azide (Carbosynth, 1.2 eq/alkyne) was added. The solution was degassed by bubbling N₂ through the solution for 20 minutes and then stirred under N₂. Separately, a solution of Cu(I) was prepared by addition of sodium ascorbate (0.50 eq/alkyne) to a degassed solution of Cu(II)SO₄ (0.10 eq/alkyne) and pentamethyldiethylenetriamine (0.10 eq/alkyne). The solution turned dark blue. The Cu(I) solution was transferred to the azide/alkyne solution via syringe. The reaction was stirred at room temperature for 48 hours and then transferred to 2000 MWCO dialysis tubing and dialyzed against 0.10 M NaCl for 24 hours, followed by dialysis against DI water for 48 hours with water changes twice per day. The contents of the dialysis tubing were then lyophilized to dryness to give the product, 5b, as a white solid (95% yield). ¹H NMR (500 MHz, D₂O, 25° C.): δ 8.13 (s, 1H), 7.49-7.31 (m, 4H), 5.73 (s, 1H), 4.59 (s, 1H), 4.49 (s, 2H), 4.30 (s, 5.5H), 4.00-3.86 (m, 2H), 3.81-3.58 (m, 7H), 3.41 (s, 1H), 3.29 (s, 1H), 3.01 (s, 11H), 2.84-2.72 (m, 3H), 2.35-2.15 (m, 22H), 1.45 (s, 11H).

### Copper(I)-catalyzed Azide-Alkyne Cycloaddition of 5-azidoacetamido-fluorescein with sulfonium 3g to give 5c

Polysulfonium 3g was dissolved in water (5 mg/mL) and 5-azidoacetamido-fluorescein 4c, (0.020 eq/alkyne, 1 eq per 3g chain) was added. The solution was degassed by bubbling N₂ through the solution for 20 minutes and then stirred under N₂. Separately, a solution of Cu(I) was prepared by addition of sodium ascorbate (0.50 eq/alkyne) to a degassed solution of Cu(II)SO₄ (0.10 eq/alkyne) and pentamethyldiethylenetriamine (0.10 eq/alkyne). The solution turned dark blue. The Cu(I) solution was transferred to the azide/alkyne solution via syringe. The reaction was covered with foil and stirred at room temperature for 48 hours and then transferred to 2000 MWCO dialysis tubing and dialyzed against 0.10 M NaCl for 24 hours, followed by dialysis against DI water for 48 hours with water changes twice per day. The contents of the dialysis tubing were then lyophilized to dryness to give the product, 5c, as a white solid (95% yield).

### Preparation of 5-azidoacetamido-fluorescein

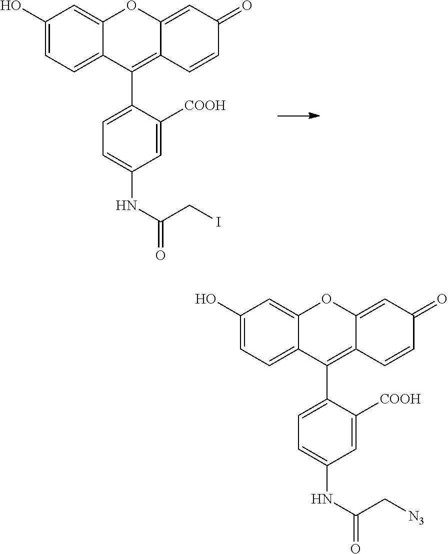 [SMILES: O=C(CI)Nc1ccc(-c2c3ccc(=O)cc-3oc3cc(O)ccc23)c(C(=O)O)c1 ; [N-]=[N+]=NCC(=O)Nc1ccc(-c2c3ccc(=O)cc-3oc3cc(O)ccc23)c(C(=O)O)c1]

5-iodoacetamido-fluorescein (5 mg, 9.70 μmol) was dissolved in DMSO (1 mL). NaN₃ (1.89 mg, 29.1 μmol, 3.00 eq) was added. The reaction was covered with foil and stirred at 50° C. for 8 hours. Water was added (20 mL) and the mixture was extracted with 3 portions of 1/1 ethyl acetate/isopropanol (10 mL each). The combined organic phases were washed with 2 small portions of brine, dried with magnesium sulfate, and condensed to give 4c as an orange solid (4.1 mg, 99%) ¹H NMR (500 MHz, d-DMSO, 25° C.): δ 10.76 (s, 1H), 10.15 (s, 1H), 8.29 (s, 1H), 7.84 (d, ³J (H, H)=7.2, 2H), 7.22 (d, ³J (H, H)=7.2, 2H), 6.66 (s, 2H), 6.58-6.51 (m, 4H), 4.13 (s, 2H). ¹³C NMR (125 MHz, CDCl₃, 25° C.): δ 169.0, 167.5, 159.9, 152.4, 147.7, 140.5, 129.49, 127.5, 127.0, 125.1, 114.2, 113.1, 110.0, 102.6, 55.3.

General Procedure for Dealkylation of Methionine Sulfonium Salts to Regenerate KM:

Alkylated KM was dissolved in 0.1 M nucleophile (2-mercaptopyridine, thiourea, mercaptoethanol, or glutathione) in PBS buffer, pH 7.4 and stirred at 37° C. At different time points, an aliquot of each reaction was removed and transferred to 2000 MWCO dialysis tubing. Samples were dialyzed against 0.10 M NaCl for 24 hours to exchange all counterions to chloride, followed by dialysis against DI water for 48 hours with water changes twice per day. Reactions with glutathione were dialyzed against 0.10 M NaCl at pH 3 for 24 hours to disrupt polyelectrolyte complexes between glutathione and the polypeptide, followed by dialysis against DI water for 48 hours with water changes twice per day. The contents of the dialysis tubing were then lyophilized to dryness.

¹H NMRs of the products of dealkylation reactions of polysulfoniums 3a, 3b, and 3e were found to be identical to the respective alkylated starting materials, and no regeneration of methionine was observed. Products of dealkylation reactions of polysulfoniums 3c, 3d, 3f, 3g, and 5a-c were found to give products with ¹H NMRs identical to the parent polypeptide KM.

A dealkylation reaction was performed using 3g and 1 eq of 2-mercaptopyridine per sulfonium group (0.02 M in DI water, 37° C.). Complete dealkylation was found to occur in 36 hours under these conditions, and to yield the parent polypeptide KM.

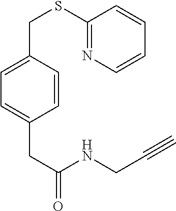 [SMILES: C#CCNC(=O)Cc1ccc(CSc2ccccn2)cc1]

Isolation of Byproduct from Dealkylation Reactions of 3g Using Mercaptopyridine:

Polysulfonium 3g was treated with 0.1 M mercaptopyridine in PBS buffer for 24 hours at room temperature. The reaction was extracted with 3 portions of ethyl acetate, and the combined organic extractions were condensed by rotary evaporation. The residue was purified by flash chromatography on silica (5% methanol in benzene) and was found to contain only excess mercaptopyridine and the expected thioether reaction byproduct. This structure was confirmed by preparation of an authentic sample by reaction of mercaptopyridine (1.5 eq) with 4-bromomethyl-N-propargyl-phenylacetamide 2g (1 eq) and K₂CO₃ (1.5 eq) in DMF for 16 hours. Spectral data were identical to the byproduct isolated from the polypeptide dealkylation reaction. ¹H NMR (500 MHz, CDCl₃, 25° C.): δ 8.46 (d, ³J (H, H)=4.9, 1H), 7.48 (dd, ³J (H, H)=8.4, 7.0 1H), 7.40 (d, ³J (H, H)=8.0, 2H), 7.18 (d, ³J (H, H)=8.2, 2H), 7.16 (s, 1H), 7.00 (dd, ³J (H, H)=6.9, 5.4 1H), 5.54 (s, 1H), 4.44 (s, 2H), 4.00 (dd, ³J (H, H)=5.3, 2.5 2H), 3.56 (s, 2H), 2.17 (t, ³J (H, H)=2.5, 1H). ¹³C NMR (125 MHz, CDCl₃, 25° C.): δ 170.5, 158.5, 149.4, 137.6, 136.0, 133.0, 129.7, 129.6, 122.2, 119.7, 79.3, 71.6, 43.2, 33.9, 29.3. HRMS-ESI (m/z) [M+H]⁺ C₁₇H₁₆N₂OS, calcd: 296.10. found: 296.10.

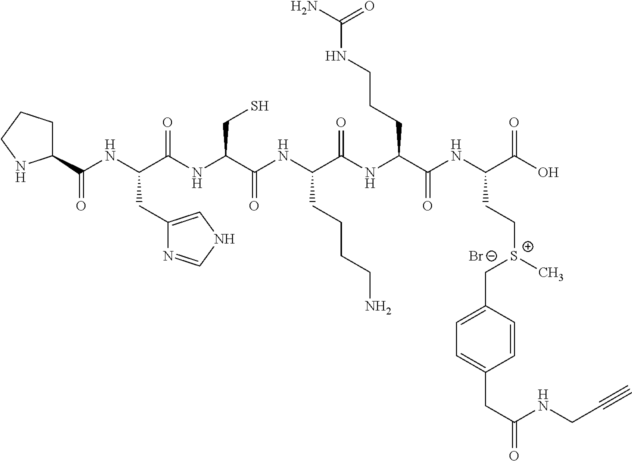 [SMILES: C#CCNC(=O)Cc1ccc(C[S+](C)CC[C@H](NC(=O)[C@H](CCCNC(N)=O)NC(=O)[C@H](CCCCN)NC(=O)[C@H](CS)NC(=O)[C@H](Cc2c[nH]cn2)NC(=O)[C@@H]2CCCN2)C(=O)O)cc1 ; [Br-]]

Alkylation of PHCKRM Peptide at PH 2.4:

PHCKRM was purchased from Bachem. PHCKRM (2.0 mg, 2.6 μmol, 1.0 eq) was dissolved in 0.2 M formic acid (0.5 mL) and 4-bromomethyl-N-propargyl-phenylacetamide 2g (0.76 mg, 2.85 μmol, 1.5 eq) was added as a 25 mg/mL solution in ethanol. The reaction was stirred for 48 hours and then extracted with 3 portions of ethyl acetate. The remaining aqueous solution was lyophilized to dryness to give 2.27 mg of 21 (92% yield), which was directly analyzed by mass spectrometry and ¹H NMR (see FIGS. 14, and 15).

Alkylation of PHCKRM at PH 8.3:

PHCKRM (1.0 mg, 1.3 μmol, 1.0 eq) was dissolved in carbonate buffer (0.25 mL) pH 8.3 and 4-bromomethyl-N-propargyl-phenylacetamide 2g (0.068 mg, 2.6 μmol, 2.0 eq) was added as a 25 mg/mL solution in ethanol. The reaction was stirred for 48 hours and then extracted with 3 portions of ethyl acetate. A sample of the remaining aqueous solution was analyzed by mass spectrometry and the remainder was lyophilized to yield a white solid. (see FIG. 16)

Dealkylation of Alkylated PHCKRM (21) Using Mercaptopyridine:

Alkylated PHCKRM 21 (2.3 mg, 2.4 μmol, 1.0 eq) was dissolved in DI water and 2-mercaptopyridine (0.78 mg, 7.1 μmol, 3 eq) was added. The solution was stirred for 24 hours at room temperature and then extracted with 5 portions of ethyl acetate. The remaining aqueous solution was lyophilized to dryness and then directly analyzed by mass spectrometry and ¹H NMR (see FIGS. 14, 15 and 17). ¹H NMR was found to be identical to the original peptide PHCKRM.

Reactivity of Cysteine, Histidine, and Lysine with Benzyl Bromide Under Acidic Conditions

As control experiments, the reactivity of N-α-CBz-cysteine, N-α-CBz-histidine, or N-α-CBz lysine with an alkylating reagent was studied.

Cysteine: N-α-CBz-cysteine (50.0 mg, 0.196 mmol, 1.00 eq) was dissolved in 1:1 THF:0.2M aqueous formic acid (2 mL), ˜pH 2.4. Benzyl bromide (67.0 mg, 0.392 mmol, 2.00 eq) was added and the reaction was covered with foil and stirred for 48 hours at room temperature. The reaction was diluted with water (30 mL), made basic with 2M NaOH, and extracted with diethyl ether (3×15 mL). The combined diethyl ether extracts were dried over magnesium sulfate and condensed to a clear oil. The aqueous phase was made acidic with concentrated HCl and extracted with EtOAc (3×15 mL). The EtOAC extracts were pooled, washed with brine, dried over magnesium sulfate, and condensed to a white solid. ¹H NMR of the diethyl ether extract was found to contain only benzyl bromide, and the EtOAc extract contained only N-α-CBz-cysteine. No alkylation occurred at pH 2.4.

Histidine: N-α-CBz-histidine (55.5 mg, 0.192 mmol, 1.00 eq) was dissolved 1:1 THF:0.2 M aqueous formic acid (2 mL), pH 2.4. Benzyl bromide (65.7 mg, 0.384 mmol, 2.00 eq) was added and the reaction was covered with foil and stirred for 48 hours at room temperature. The reaction was extracted with diethyl ether (3×15 mL). The combined diethyl ether extracts were dried over magnesium sulfate and condensed to a clear oil. The aqueous phase was lyophilized to dryness. ¹H NMR of the diethyl ether extract was found to contain only benzyl bromide, and ¹H NMR of the aqueous portion contained only N-α-CBz-histidine. No alkylation occurred at pH 2.4.

Lysine: N-α-CBz-lysine (0.0500 mg, 0.178 mmol, 1.00 eq.) was treated with benzyl bromide (61.0 mg, 0.357 mmol, 2.00 eq.) as previously described for N-α-CBz-histidine. ¹H NMR of the diethyl ether extract was found to contain only benzyl bromide, and ¹H NMR of the aqueous portion contained only N-α-CBz-lysine. No alkylation occurred at pH 2.4.

Experiments to Check for Chain Cleavage Resulting from Alkylation or Dealkylation Reactions:

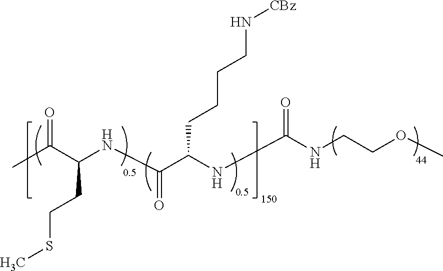

### Poly[(N_{ε}-carbobenzyloxy-L-lysine)_{0.5}-stat-(L-methionine)_{0.5}]₁₅₀-block-poly(ethylene glycol)₂₂, 22

Inside a dinitrogen filled glove box, a solution of N_{ε}-carbobenzyloxy-L-lysine-N-carboxyanhydride (21) (Cbz-Lys NCA), (25 mg, 0.082 mmol, 1 eq) and Met NCA (14 mg, 0.082 mmol, 1.0 eq) in dry THF (0.15 M) was prepared. A solution of (PMe₃)₄Co in dry THF (20 mM) was rapidly added via syringe (1.5 mg, 4.1 μmol, 0.025 eq). After 45 min, the polymerization reaction was complete as determined by FTIR. An aliquot was removed and analyzed by GPC/LS. A solution of α-methoxy-ω-isocyanoethyl-poly(ethylene glycol) (42) (PEG-NCO, MW=2,000) in THF (12 mg, 0.012 mmol, 3.0 eq per (PMe₃)₄Co) was added to the polymerization reaction. The reaction immediately turned from pale orange to green and was stirred overnight at room temperature. The reaction was then removed from the glove box and HCl (6 M in H₂O, 2.0 eq. per (PMe₃)₄Co) was added to the solution, which turned a blue-green color. After 10 min stirring, the PEG-endcapped copolypeptide was collected by precipitation into water (pH 3, HCl, >10× the reaction volume), followed by centrifugation. The white solids were washed with 3 portions of DI water to remove all unconjugated PEG-NCO, collected by centrifugation, and lyophilized to give 44 mg of a white solid (99% yield). Since it has been shown that end-capping is quantitative for (PMe₃)₄Co initiated NCA polymerizations when excess isocyanate is used (7), integrations of copolypeptide resonances versus the polyethylene glycol resonance at δ 3.64 could be used to obtain copolypeptide lengths. Mₙ=29,490, M_{w}/Mₙ=1.14, DP=150. ¹H NMR (500 MHz, CDCl₃ with 1% d-TFA, 25° C.): δ 8.16 (br s, 2H), 7.31 (s, 5H), 5.11 (s, 2H), 4.19 (s, 1H), 3.95 (s, 1H), 3.75 (s, 1.18), 3.14 (s, 2H), 2.74-2.47 (m, 2H), 2.31-1.77 (m, 7H), 1.60-1.29 (br m, 4H). (see FIG. 18).

Reactivity of 2-Mercaptopyridine with Disulfide Bonds

2-Mercaptopyridine (66.7 mg, 0.600 mmol, 3.00 eq) was added to a solution of cystine (48.0 mg, 0.200 mmol, 1.00 eq) in DI water (5 mL). The solution was stirred for 24 hours at 37° C. Water was added (3 mL) and the aqueous solution was extracted with EtOAc (3×5 mL). The aqueous phase was lyophilized to dryness and analyzed by ¹H NMR and ¹³C NMR. Spectral data was identical to that of an authentic sample of cystine, no disulfide reduction was observed.

Stability of Polysulfoniums:

PBS Buffer:

3c and 3g were dissolved in PBS buffer (10 mg/mL) and were maintained at room temperature for 2 weeks. Samples were then transferred to 2000 MWCO dialysis tubing, dialyzed against DI water for 16 hours, then lyophilized to dryness. ¹H NMR spectra were identical to spectra of the parent copolypeptides. Homopolymers of (S-methyl-L-methionine sulfonium chloride) and (S-carboxymethyl-L-methionine sulfonium chloride) were previously shown to be stable in water, DMF, PBS buffer, or DMEM cell culture media for >1 week at room temperature (42).

Storage as Dry Solids:

All polysulfoniums described were found to be stable for >6 months when stored as dry powders at room temperature, with the exception of 3d. After ca. 4 weeks of storage as a dry solid copolypeptide 3d becomes very difficult to redissolve in previously good solvents (water, TFA).

Base:

Water was made basic to pH 10.1 by addition of drops of 0.5 M NaOH. 3c, 3d, and 3g were each dissolved in pH 10.1 water at a concentration of 1 mg/mL. The solutions were allowed to stand at room temperature for 10 hours and then transferred to 2000 MWCO dialysis tubing. The solutions were then dialyzed against DI water for 16 hours, and then the contents of the dialysis tubing were lyophilized to dryness. ¹H NMR spectra of 3c, and 3g before and after treatment with base were identical. ¹H NMR spectra of 3d showed complete dealkylation to give the parent KM. Treatment of 3d with aqueous bicarbonate at pH 9.8 also resulted in complete dealkylation to give the parent KM. Homopolymers of (S-methyl-L-methionine sulfonium chloride) and (S-carboxymethyl-L-methionine sulfonium chloride) were previously shown to be stable in water at pH 10 (NaOH) for >3 hours (42).

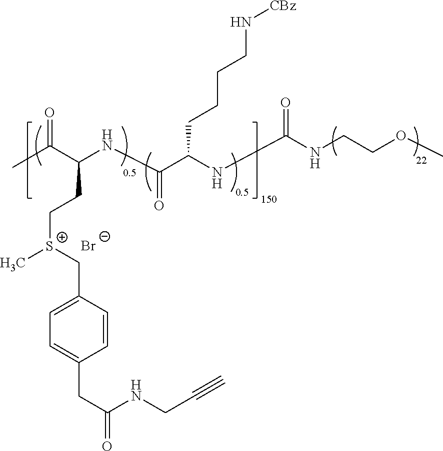

### Poly[(N_{ε}-carbobenzyloxy-L-lysine)_{0.5}-stat-(S-(4-(N-propargyl-acetamido)phenylmethyl)-L-methionine sulfonium bromide_{0.5}]₁₅₀-block-poly(ethylene glycol)₂₂, 23

22 (44.1 mg, 1.45 μmol) was dissolved in DMF (3 mL). 4-Bromomethyl-N-propargyl-phenylacetamide 2g (28.9 mg, 0.109 mmol, 2.00 eq per methionine residue) was added. The reaction mixture was covered with foil and stirred at room temperature for 48 hours. Diethyl ether was added (20 mL) and the white precipitate was collected by centrifugation. The precipitate was washed with 2 portions of dichloromethane and then dried under high vacuum to give 77.4 mg (99%). A solution of the alkylated copolypeptide was prepared in 0.1M LiBr in DMF (5 mg/mL) and analyzed by GPC/LS. ¹H NMR showed no change in the ratio of PEG to polypeptide. Mₙ=53,750; M_{w}/Mₙ=1.18. (see FIG. S8). ¹H NMR (500 MHz, d-TFA, 25° C.): δ 7.59-7.35 (m, 4H), 7.26 (s, 5H), 6.78 (br s, 1H), 5.15 (s, 2H), 4.80 (s, 1H), 4.65 (s, 2H), 4.52 (s, 1H), 4.15-3.98 (m, 3H), 3.86 (s, 4.02), 3.71 (s, 2H), 3.19 (s, 2H), 3.00 (s, 2H), 2.83-2.55 (m, 2H), 2.25-2.0 (m, 4H), 1.99-1.68 (m, 4H), 1.67-1.30 (m, 4H).

Dealkylation of Sulfonium Groups in 23

23 (77.4 mg, 1.45 μmol) was dissolved in DMF (3 mL). 2-Mercaptopyridine (12.0 mg, 0.109 mmol, 2.00 eq per methionine residue) was added. The reaction mixture was stirred at room temperature for 24 hours. Diethyl ether was added (20 mL) and the white precipitate was collected by centrifugation. The precipitate was washed with 2 more portions of diethyl ether and then dried under high vacuum to give the product 43.9 mg (98%). A solution of the copolypeptide was prepared in 0.1M LiBr in DMF (5 mg/mL) and analyzed by GPC/LS. GPC/LS and ¹H NMR spectra were identical to the parent copolypeptide 22. (see FIG. 18).

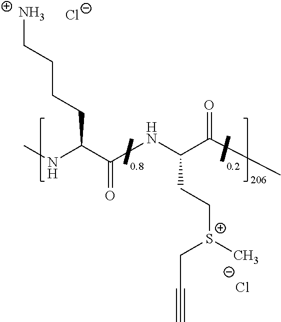

### Poly[(L-lysine.HCl)_{0.8}-stat-(S-propargyl-L-methionine sulfonium chloride)_{0.2}]₂₀₆, 24

Route 1: Deprotection of lysine residues followed by methionine alkylation

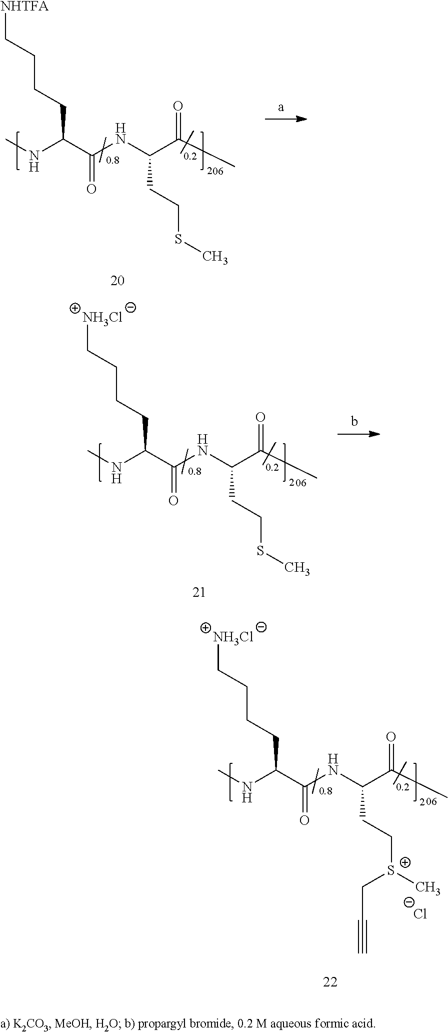

Poly[(N_{ε}-TFA-L-lysine)_{0.8}-stat-L-methionine)_{0.2}]₂₀₆ was dispersed in methanol:water, 9:1 (20 mg/mL) and K₂CO₃ (2 eq per lysine residue) was added. The reaction was stirred for 8 hours at 50° C. and then the methanol was removed by rotory evaporation. The remaining solution was then diluted with water and transferred to a 2000 MWCO dialysis bag, and dialyzed against 0.10 M NaCl at pH 3 (HCl) for 24 hours, followed by DI water for 48 hours with water changes twice per day. The contents of the dialysis bag were then lyophilized to dryness to give a white solid, 25. ¹H NMR (500 MHz, D₂O, 25° C.): δ 4.53-4.45 (m, 0.25H), 4.32 (br s, 1H), 3.03-2.99 (m, 0.45H), 2.10-1.97 (m, 1.18H), 1.85-1.67 (br m, 4H), 1.52-1.37 (br m, 2H).

The resulting poly[(L-lysine.HCl)_{0.8}-stat-(L-methionine)_{0.2}]₂₀₆, 25, was next reacted with propargyl bromide using alkylation method A in 0.2 M aqueous formic acid to give the final product, 24.

Route 2: Methionine alkylation followed by deprotection of lysine residues

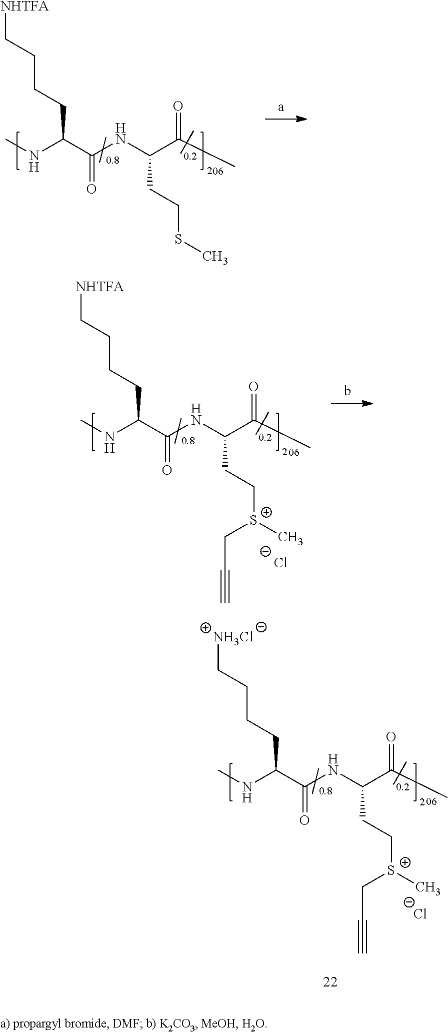

Poly[(N_{ε}-TFA-L-lysine)_{0.8}-stat-(L-methionine)_{0.2}]₂₀₆ was dissolved in DMF (20 mg/mL) and propargyl bromide was added (3 eq per methionine residue). The solution was covered with foil and stirred for 48 hours at room temperature. The polymer was precipitated with ether and the solids collected by centrifugation. The solids were washed with 2 more portions of ether and then dried under high vacuum. Poor solubility of this copolymer prevented collection of a meaningful NMR spectrum.

The TFA protecting groups of the resulting poly[(N_{ε}-TFA-L-lysine)_{0.8}-stat-(S-propargyl-L-methionine sulfonium chloride)_{0.2}]₂₀₆ were next removed using the same procedure as described under Route 1 above to give the final product, 24.

The ¹H NMR spectra of the final alkylated copolymers prepared using either of the two routes above were found to be identical. ¹H NMR (500 MHz, D₂O, 25° C.): δ 4.67-4.56 (m, 0.25H), 4.33 (br s, 1H), 3.60-3.41 br m, 0.48H), 3.20-2.95 (m, 3.24H), 2.47-2.22 (m, 0.77H), 1.90-1.67 (br m, 4H), 1.50 (br s, 2H). ¹⁹F NMR (400 MHz, D₂O, 25° C.): no signals.

Reactivity of Poly(L-Lysine.HCl) with Propargyl Bromide Under Acidic Conditions

As a control experiment, poly(L-lysine.HCl) was dissolved in 0.2M aqueous formic acid (20 mg/mL). Propargyl bromide was added (2 eq) and the reaction was covered with foil and stirred for 36 hours. The reaction was transferred to a 2000 MWCO dialysis bag, dialyzed against DI water for 72 hours with water changes twice per day, and then lyophilized to give a white solid. ¹H NMR spectrum was identical to the starting poly(L-lysine.HCl), no alkylation was observed.

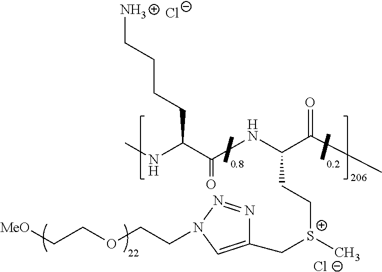

### Poly[(L-lysine.HCl)_{0.8}-stat-(S-(1-poly(ethylene glycol)₄₄-1,2,3-triazolylmethyl-L-methionine sulfonium chloride)_{0.2}]₂₀₆, 26

CuSO₄ (0.05 eq per alkyne) was dissolved in water and sodium ascorbate (0.25 eq per alkyne) was added followed by PMDETA (0.1 eq per alkyne). The dark blue solution was stirred under N₂ for 15 min and then added to a solution of poly[(L-lysine.HCl)_{0.8}-stat-(S-propargyl-L-methionine sulfonium chloride)_{0.2}]₂₀₆ and α-methoxy-ω-azidoethyl-poly(ethylene glycol) (1.5 eq per alkyne, Mₙ=1000 Da, Sigma Aldrich). The solution was degassed by placing under partial vacuum and backfilling with N₂. The reaction was stirred for 24 hours at room temperature and then transferred to an 8000 MWCO dialysis bag. The reaction was dialyzed against 0.10 M NaCl for 24 hours, followed by DI water for 72 hours with water changes twice per day. The contents of the dialysis bag were then lyophilized to dryness to give the product as a white solid (93% yield, 100% pegylation as determined by NMR). ¹H NMR (500 MHz, D₂O, 25° C.): δ 4.56-4.49 (m, 0.25H), 4.32 (br s, 1H), 3.72 (s, 22H), 3.67-3.62 (m, 0.48H), 3.54-3.49 (m, 0.25H), 3.40 (s, 0.48H), 3.02 (br s, 2.75H), 2.64-2.52 (m, 0.49H), 1.88-1.64 (br m, 4H), 1.47 (br s, 2H).

TFA-Lysine NCA

To a solution of N_{ε}-TFA-L-lysine (1.00 g, 4.13 mmol) in dry THF (0.15 M) in a Schlenk flask was added a solution of phosgene in toluene (8.26 mmol, 20% (w/v), 2 eq) via syringe. Caution! Phosgene is extremely hazardous and all manipulations must be performed in a well-ventilated chemical fume hood with proper personal protection and necessary precautions taken to avoid exposure. The reaction was stirred under N₂ at 50° C. for 3 hrs. The reaction was evaporated to dryness and transferred to a dinitrogen filled glove box. The condensate in the vacuum traps was treated with 50 mL of concentrated aqueous NH₄OH to neutralize residual phosgene. The NCA was purified by recrystallization in dry THF/hexanes to give 0.940 g (85% yield) of the product as a white solid. Spectral data was in agreement with previously published results (29).

### Stability of poly(Met) sulfonium salts

In general, poly(Met) sulfonium salts prepared in this study were stable for >3 months when stored as solids at room temperature (poly(S-(2-bromoethyl)-L-methionine sulfonium triflate) was the only sample stored at −20° C.). Aqueous solutions of poly(S-methyl-L-methionine sulfonium chloride), 3, or poly(S-carboxymethyl-L-methionine sulfonium chloride), 4, (10 mg/mL) were subjected to various conditions to evaluate stability. Solutions of these polymers were heated at 80° C. for 16 hours, or stored for 3 hours at pH 2 (HCl), pH 10 (NaOH), or in 0.5M NaCl. Solutions of 3 or 4 (10 mg/mL) in water, DMF, PBS buffer, or DMEM cell culture media were stable for >1 week at room temperature. ¹H NMR spectra of samples after being subjected to each of these various conditions were identical to the starting materials and no polymer precipitation was observed.

The following claims are thus to be understood to include what is specifically illustrated and described above, what is conceptually equivalent, what can be obviously substituted and also what essentially incorporates the essential idea of the invention. Those skilled in the art will appreciate that various adaptations and modifications of the just-described preferred embodiment can be configured without departing from the scope of the invention. The illustrated embodiment has been set forth only for the purposes of example and that should not be taken as limiting the invention. Therefore, it is to be understood that, within the scope of the appended claims, the invention may be practiced other than as specifically described herein. Cited publications are incorporated herein by reference to the extent permitted by rule or statute.

### REFERENCES

1. Angelos, S.; Yang, Y-W.; Patel, K.; Stoddart, J. F.; Zink, J. I. Angew. Chem. Int. Ed. 2008, 47, 2222-2226.
2. Augspurger, N. R.; Scherer, C. S.; Garrow, T. A.; Baker, D. H. J. Nutrition 2005, 135, 1712-1717.
3. Barner-Kowollik, C.; Du Prez, F. E.; Espeel, P.; Hawker, C. J.; Junkers, T.; Schlaad, H.; Van Camp, W. Angew. Chem. Int. Ed. 2011, 50, 60-62.
4. Bittner, E. W., J. T. Gerig, J. Amer. Chem. Soc., 1970, 92, 2114-2118.
5. Bodanszky, M., M. A. Bednarek, Int. J. Peptide Protein Res. 1982, 20, 408-413.
6. Bradbury, J. H.; Chapman, B. E. Aust. J. Chem. 1970, 23, 1801-1809.
7. Brzezinska, K. R.; Curtin, S. A.; Deming, T. J. Macromolecules, 2002, 35, 2970-2976.
8. Chalker, J. M., G. J. L. Bernardes, B. G. Davis, Acc. Chem. Res. 2011, 44, 730-741.
9. Chalker, J. M., G. J. L. Bernardes, Y. A. Lin, B. G. Davis, Chem. Asian J. 2009, 4, 630-640.
10. Chen, C.; Wang, Z.; Li, Z. Biomacromolecules 2011, 12, 2859-2863.
11. Cheng, J.; Deming, T. J. Top. Curr. Chem. 2012, 310, 1-26.
12. Cheng, Y.; He, C.; Xiao, C.; Ding, J.; Zhuang, X.; Chen, X. Polym. Chem. 2011, 2, 2627-2634.
13. Cohen, L. H., Annu. Rev. Biochem. 1968, 37, 695-726.
14. Deming, T. J. Macromolecules 1999, 32, 4500-4502.
15. Deming, T. J. Nature 1997, 390, 386-389.
16. Doi, J. T., G. W. Luehr, Tet. Lett. 1985, 26, 6143-6146.
17. Engler, A. C.; Lee, H.-I; Hammond, P. T. Angew. Chem. Int. Ed. 2009, 48, 9334-9338.
18. Foettinger, A., A. Leitner, W. Lindner, J. Chromatog. A 2005, 1079, 187-196.
19. Foettinger, A., A. Leitner, W. Lindner, J. Proteome Res. 2007, 6, 3827-3834.
20. Franzen, V.; Schmidt, H.-J.; Mertz, C. Chem. Ber. 1962, 94, 2942-2950.
21. Fuller, W. D.; Verlander, M. S.; Goodman, M. Biopolymers 1976, 15, 1869-1871.
22. Gauthier, M. A.; Gibson, M. I.; Klok, H.-A. Angew. Chem. Int. Ed. 2009, 48, 48-58.
23. Glazer, A. N., Annu. Rev. Biochem. 1970, 39, 101-130.
24. Gobom, J., M. Schuerenberg, M. Mueller, D. Theiss, H. Lehrach, E. Nordhoff, Anal. Chem. 2001, 738, 434-438.
25. Goren, H. J., E. A. Barnard, Biochemistry 1970, 9, 959-973.
26. Guillermain, C.; Gallot, B. Macromol. Chem. Phys. 2002, 203, 1346-1356 (TFA-Lys NCA spectral data)
27. Guinn, R. M.; Margot, A. O.; Taylor, J. R.; Schumacher, M.; Clark, D. S.; Blanch, H. W. Biopolymers 1994, 35, 503-512.
28. Gundlach, H. G., S. Moore, W. H. Stein, J. Biol. Chem. 1959, 234, 1761-1764.
29. Gundlach, H. G., W. H. Stein, S. Moore, J. Biol. Chem. 1959, 234, 1754-1760.
30. Habraken, G. J. M.; Koning, C. E.; Heuts, J. P. A.; Heise, A. Chem. Commun. 2009, 45, 3612-3614.
31. Hoffmann, H. M. R., E. D. Hughes, J. Chem. Soc., 1964, 1252-1258.
32. Howells, R. D.; Mc Cown, J. D. Chem. Rev. 1977, 77, 69-92.
33. Huang, J.; Habraken, G. J. M.; Audouin, F.; Heise, A. Macromolecules 2010, 43, 6050-6057.
34. Huang, Y.; Zeng, Y.; Yang, J.; Zeng, Z.; Zhu, F.; Chen, X. Chem. Commun. 2011, 47, 7509-7511.
35. Jones, J. B., D. W. Hysert, Can. J. Chem. 1971, 49, 3012-3019.
36. Kawai, T.; Komoto, T. J. Cryst. Growth 1980, 48, 259-282.
37. Kiessling, L. L.; Gestwicki, J. E.; Strong, L. E. Angew. Chem. Int. Ed. 2006, 45, 2348-2368.
38. Kleanthous, C. J. R. Coggins, J. Biol. Chem. 1990, 265, 10935-10939.
39. Klein, H. F.; Karsch, H. H. Chem. Ber. 1975, 108, 944-955.
40. Kobayashi, H.; Nakashima, K.; Ohshima, E.; Hisaeda, Y.; Hamachi, I.; Shinkai, S. J. Chem. Sci., Perkin Trans. 2, 2000, 997-1002.
41. Kramer, J. R.; Deming, T. J. Biomacromolecules 2010, 11, 3668-3672.
42. Kramer, J. R.; Deming, T. J. Biomacromolecules 2012, 13, 1719-1723;
43. Kramer, J. R.; Deming, T. J. Biomacromolecules, 2010, 11, 3668-3672.
44. Kramer, J. R.; Deming, T. J. J. Amer. Chem. Soc., 2010, 132, 15068-15071.
45. Kramer, J. R., T. J. Deming, Biomacromolecules, 2012, 13, 1719-1723.
46. Kussman, M., E. Nordhoff, H. Rahbek-Nielsen, S. Haebel, M. Rossel-Larsen, L. Jakobsen, J. Gobom, E. Mirgorodskaya, A. Kroll-Kristensen, L. Palm, P. Roepstorff, J. Mass Spectrom. 1997, 32, 593-601.
47. Landis, B. H., L. J. Berliner, J. Amer. Chem. Soc., 1980, 102, 5350-5354.
48. Lin, W-C., T. H. Morton, J. Org. Chem. 1991, 56, 6850-6856.
49. Lindorff-Larson, K., J. R. Winther, Anal. Biochem. 2000, 286, 308-310.
50. Lu, H.; Bai, Y.; Wang, J.; Gabrielson, N. P.; Wang, F.; Lin, Y. Cheng, J. Macromolecules 2011, 44, 6237-6240. (e) Tang, H.; Zhang, D. Polym. Chem. 2011, 2, 1542-1551.
51. Makino, S.; Sugai, S. J. Poly. Sci., Part A-2 1967, 5, 1013-1028.
52. McAvey, K. M., B. Guan, C. A. Fortier, M. A. Tarr, R. B. Cole, J. Am. Soc. Mass Spectrom. 2011, 22, 659-669.
53. McRorie, R.; Sutherland, G. L.; Lewis, M. S.; Barton, A. D.; Glazener, M. R.; Shive, W. J. Amer. Chem. Soc. 1954, 76, 115-118.
54. Mohan, S.; Sim, L.; Rose, D. R.; Pinto, B. M. Carbohydrate Res. 2007, 342, 901-912.
55. Nagasaki, T.; Kimura, T.; Arimori, S.; Shinkai, S. Chem. Lett. 1994, 1495-1498.
56. Naider, F., Z. Bohak, Biochemistry 1972, 11, 3208-3211.
57. Naider, F., Z. Bohak, J. Yariv, Biochemistry 1972, 11, 3202-3208.
58. Nessen, M. A., G. Kramer, J. W. Back, J. M. Baskin, L. E. J. Smeenk, L. J. de Koning, J. H. van Maarseveen, L. de Jong, C. R. Bertozzi, H. Hiemstra, C. G. de Koster, J. Proteome Res. 2009, 8, 3702-3711.
59. Noble, R. L., D. Yamashiro, C. H. Li, J. Amer. Chem. Soc., 1976, 98, 2324-2328.
60. Noguchi, J.; Tokura, S.; Nishi, N. Angew. Makromol. Chem. 1972, 22, 107-131.
61. Olejnik, J., S. Sonar, E. Krzymanska-Olejnik, K. J. Rothschild, Proc. Natl. Acad. Sci. USA 1995, 92, 7590-7594.
62. Park, K. D., R. Liu, H. Kohn, Chem. & Biol. 2009, 16, 763-772.
63. Pasut, G., F. M. Veronese Adv. Drug Deliv. Rev. 2009, 61, 1177-1188
64. Perlman, G. E., E. Katchalski, J. Amer. Chem. Soc. 1962, 84, 452-457.
65. Poché D. S.; Thibodeaux, S. J.; Rucker, V. C.; Warner, I. M.; Daly, W. H. Macromolecules 1997, 30, 8081-8084.
66. Prescher, J. A., C. R. Bertozzi, Nat. Chem. Biol. 2005, 1, 13-21.
67. Satoyoshi, D.; Hachisu, M; Amaike, M.; Ohkawa, K.; Yamamoto, H. Macromol. Mater. Eng. 2004, 289, 495-498.
68. Schramm, H. J., W. B. Lawson, Zeitschrift f. Physiol. Chem. 1963, 332, 97-100.
69. Slavin, S.; Burns, J.; Haddleton, D. M.; Becer, C. R. Eur. Polym. J. 2011, 47, 435-446.
70. Sousa-Herves, A.; Fernandez-Megia, E.; Riguera, R. Chem. Commun. 2008, 3136-3138.
71. Sun, J.; Schlaad, H. Macromolecules 2010, 43, 4445-4448.
72. Szychowski, J., A. Mandavi, J. J. L. Hodes, J. D. Bagert, J. T. Ngo, P. Landgraf, D. C. Dieterich, E. M. Schuman, D. A. Tirrell, J. Amer. Chem. Soc., 2010, 132, 18351-18360.
73. Taichi, M., T. Kimura, Y. Nishiuchi, Int. J. Pept. Res. Ther. 2009, 15, 247-253.
74. Tang, H.; Zhang, D. Biomacromolecules 2010, 11, 1585-1592.
75. Toennies, G. J. Biol. Chem. 1940, 132, 455-456.
76. Toennies, G.; Kolb, J. J. J. Amer. Chem. Soc. 1945, 67, 849-851.
77. Vallee, B. L., J. F. Riordan, Annu. Rev. Biochem. 1969, 38, 733-794.
78. Verhelst, S. H. L., M. Fonovie, M. Bogyo, Angew. Chem. Int. Ed. 2007, 46, 1284-1286.
79. Vithayathil, P. J., F. M. Richards, J. Biol. Chem. 1960, 235, 2343-2351.
80. Yu, M.; Nowak, A. P.; Pochan, D. J.; Deming, T. J. J. Am. Chem. Soc. 1999, 121, 12210-12211.
81. Zhou, H., J. A. Ranish, J. D. Watts, R. Aebersold, Nat. Biotech. 2002, 19, 512-515.

1. A process for chemically modifying polypeptides by alkylation of thioether groups comprising the steps of:
suspending an original polypeptide containing at least one thioether containing residue in an aqueous or polar organic solvent;
adding an alkyl halide, wherein the halide is chlorine, bromine or iodine; and
reacting whereby the at least one thioether group is functionalized by the addition of an alkyl group thereby creating at least one sulfonium ion.

2. The process of claim 1, wherein where the alkyl group of the alkyl halide is selected from the group consisting of propargyl, carbamidomethyl, N-alkyl carbamidomethyl, N-aryl carbamidomethyl, O-alkyl carboxymethyl, O-(4-nitrophenyl) carboxymethyl, O—(N-succinimidyl) carboxymethyl, 2-pyridylmethyl, 3-pyridylmethyl, 4-pyridylmethyl, 2-boroxyphenylmethyl, 3-boroxyphenylmethyl, 4-boroxyphenylmethyl and mixtures of the same.

3. A process for chemically modifying polypeptides by alkylation of thioethers groups comprising the steps of:
suspending a polypeptide containing at least one thioether group in a polar organic solvent;
adding an alkyl triflate; and
reacting whereby the at least one thioether group is functionalized by the addition of the alkyl group thereby creating a sulfonium ion.

4. The process of claim 3, wherein the alkyl group of the alkyl triflate is selected from the group consisting of allyl, 2-(2-methoxyethoxy)ethyl, 2-methoxyethyl, 2-(2-methyl-1,3-dioxolyl)ethyl, 2-(2,3,4,6-tetra-O-acetyl-α-D-glucopyranosyl)oxyethyl, 3-azidopropyl, 2-(2,3,4,6-tetra-O-acetyl-α-D-galactopyranosyl)ethyl, 2-bromoethyl and mixtures of the same.

5. A process for chemically modifying polypeptides by alkylation of thioether groups comprising the steps of:
suspending a polypeptide containing at least one thioether group in a polar organic solvent;
adding an alkyl halide, wherein the halide is chlorine, bromine or iodine;
adding a solution of a silver salt; and
reacting whereby the at least one thioether group is functionalized by the addition of the alkyl group thereby creating a sulfonium ion.

6. The process of claim 5, wherein the silver salt is silver tetraborate.

7. The process of claim 5, wherein the alkyl group is selected from the group consisting of allyl, 2-(2-methoxyethoxy)ethyl, 2-methoxyethyl, 2-(2-methyl-1,3-dioxolyl)ethyl, 2-(2,3,4,6-tetra-O-acetyl-α-D-glucopyranosyl)oxyethyl, 3-azidopropyl, 2-(2,3,4,6-tetra-O-acetyl-α-D-galactopyranosyl)ethyl, and mixtures of the same.

8. The process of claim 1 further comprising a step functionalizing the at least one sulfonium ion by reacting an added chemical reagent with a functional group present on the alkyl group.

9. The process of claim 8, wherein the functional group is selected from the group consisting of alkyne, azide, alkene, ketone, aldehyde, alkyl halide, amine, ester, isocyanate; and the chemical reagent contains a reactive group selected from the group consisting of alkyne, azide, alkene, thiol, amine, hydrazine, oxyamine, and carboxylic ester.

10. The process of claim 1 further comprising a step of adding a polymerizable monomer able to react with a functionality present on the alkyl group and reacting whereby a functional group on the at least one sulfonium ion is able to initiate growth of a polymer chain.

11. The process of claim 10, wherein the functionality is selected from the group consisting of alkyl halide, alkyltrithiocarbonate, benzodithioate, N,N-dialkyl-O-alkyl hydroxylamine; and the monomer is selected from the group consisting of acrylate, methacrylate, acrylamide, methacrylamide, styrene, cyanoacrylate, acrylic acid, methacrylic acid, vinyl acetate, N-vinyl pyrrolidone, and N-vinylcarbazole.

12. The process of claim 1 further comprising a step of adding a nudeophile that reacts with the at least on sulfonium ion whereby the alkyl is removed and the original polypeptide is regenerated.

13. The process of claim 12, wherein the nudeophile is selected from the group consisting of 2-mercaptopyridine, thiourea, 4-mercaptopyridine, glutathione, 2-mercaptoethanol, 2-aminoethanethiol, thioglycolid acid, dithiothreitol and mixtures of the same.

14. The process of claim 12, wherein the alky group is an aromethyl or a carboxymethyl group.

15. The process of claim 12, wherein the aromethyl group is selected from the group consisting of (N-propargyl-4-carbamidomethyl)-phenylmethyl, (N-azidoethyl-4-carboxamido)-phenylmethyl, propargyl, benzyl, allyl, O-alkyl carboxymethyl, O-(4-nitrophenyl) carboxymethyl, and O—(N-succinimidyl) carboxymethyl.

16. The process of claim 1, wherein the at least one thioether containing residues is selected from the group consisting of methionine, S-methyl-cysteine, S-ethyl-cysteine, S-allyl-cysteine, S-benzyl-cysteine, S-farnesyl-cysteine, S-propargyl-cysteine, S-(galactopyranosyl propyl)-cysteine, S-)glucopyranosyl propyl-cysteine, S-(mannopyranosyl propyl)-cysteine, S-methyl-homocysteine, S-ethyl-homocysteine, S-allyl-homocysteine, S-benzyl-homocysteine, S-farnesyl-homocysteine, S-propargyl-homocysteine, S-(galactopyranosyl propyl)-homocysteine, S-(glucopyranosyl propyl)-homocysteine, S-(mannopyranosyl propyl)-homocysteine and mixtures of the same.
